(19) United States
(12) Patent Application Publication
(10) Pub. No.: US 2011/0166161 A1
(43) Pub. Date: Jul. 7, 2011

(54) HETEROCYCLIC CARBOXAMIDE COMPOUNDS

(75) Inventors: Takeshi Terasawa, Tokyo (JP); Shinji Shigenaga, Tokyo (JP); Shinji Itoh, Tokyo (JP); Jun Maeda, Tokyo (JP); Hideyuki Watanabe, Tokyo (JP); Satoshi Kubo, Tokyo (JP); Noe Iahii, Noe Ishii, Ibaragi (JP)

(73) Assignee: Astellas Pharma Inc., Tokyo (JP)

(21) Appl. No.: 13/061,855

(22) PCT Filed: Sep. 17, 2009
(86) PCT No.: PCT/JP09/66840

§ 371 Date: Mar. 2, 2011

Related U.S. Application Data
(60) Provisional application No. 61/097,979, filed on Sep. 18, 2008.

(51) Int. Cl. A61K 31/4965; C07D 213/72; A61K 31/44; C07D 409/02; A61K 31/4436; C07D 401/02; A61K 31/4545; C07D 241/10; C07D 239/02; A61K 31/505; A61P 19/02; A61P 9/00; A61P 11/00; A61P 27/02

(52) U.S. Cl. 514/255.06; 546/297; 514/349; 546/280.1; 514/336; 546/193; 514/318; 544/406; 544/323; 514/275; 546/309; 514/352

(57) ABSTRACT

This invention relates to novel heterocyclic carboxamide derivatives and salts thereof. More particularly, it relates to novel heterocyclic carboxamide derivatives and salts thereof which act as a ROCK inhibitor, to a pharmaceutical composition comprising the same and to a method of using the same therapeutically in the treatment and/or prevention of ROCK-related disease.

### TECHNICAL FIELD

This invention relates to novel heterocyclic carboxamide derivatives and salts thereof which are useful as a ROCK inhibitor.

### BACKGROUND ART

Rho kinases (ROCK) are serine/threonine kinases that function in downstream of Rho which is a low molecular GTP-binding protein, and two ROCK isoform, ROCK I and ROCK II, has been identified. The enzymes are involved in a variety of biological events such as cytoskeletal control, cell growth, cell migration, apoptosis, inflammation, etc. To date, it has been reported that the enzymes are involved in pathology of circulatory system disease, tumor infiltration, osteogenesis, neurodegenerative disorders, chronic inflammatory diseases, etc (see, e.g., Satoh H. et al, Jpn. J. Pharmacol. 79, Suppl I, 211 (1999), Kuwahara K. et al, FEBS Lett 452, 314-318 (1999), Sawada N. et al, Circulation 101, 2030-2033 (2000), Kataoka C. et al, Hypertension. 2002 February; 39(2): 245-50, Imamura F. et al, Jpn. J. Cancer Res. 91, 811-16 (2000), Itoh K. et al, Nat. Med. 5, 221-5 (1999), Nakajima M. et al, Clin. exp. Pharmacol. Physiol. 30, 457-63 (2003), Sun et al, J. Neuroimmunol. 180, 126-134 (2006), Salminen et al, Biochem. Biophys. Res. Commun. 371, 587-590 (2008), Ikeda et al, Am. J. Physiol. 293, G911-917 (2007), Kolavennu et al, Diabetes 57, 714-723 (2008), Schaafsma et al, Eur. J. Pharmacol. 585, 398-406 (2008)), and recently the involvement of the enzymes in chondrocyte differentiation and neurogenic pains etc has been investigated (see, e.g., Guoyan W. et al, J. Biol. Chem. 279, 13205-13214 (2004), Tatsumi S, Neuroscience. 131, 491-498 (2005), Ito et al, Spine, 32, 2070-2075 (2008)).

With elucidation of such many functions of ROCK in the body, many compounds which can inhibit the functions of the enzymes (ROCK inhibitors) has been studied extensively (see, e.g., WO/98/06433, WO00/09162, WO00/78351, WO01/17562, WO02/076976, EP1256574, WO02/100833, WO03/082808, WO04/09555, WO04/24717, WO04/108724, WO05/03101, WO05/35501, WO05/35503, WO05/35506, WO05/58891, WO05/74642, WO05/74643, WO05/80934, WO05/82367, WO05/82890, WO05/97790, WO05/100342, WO05/103050, WO05/105780, WO05/108397, WO06/09889, WO06/44753, WO06/51311, WO06/57270, WO06/58120, WO06/71458, WO06/72792, WO06/105081, WO06/135383, WO06/136821, WO07/00240, WO07/06546, WO07/06547, WO07/12421, WO07/12422, WO07/26664, WO07/42321, WO07/60028, WO07/65916, WO07/84667, WO07/125,315, WO07/125,321, WO07/142,323, WO08/20081, WO08/11557, WO08/11560, WO08/20081, WO08/36540, WO08/49919, WO08/54599, WO08/77057, WO08/77550, WO08/77551, WO08/77552, WO08/77553, WO08/77554, WO08/77555, WO08/77556, WO WO08/79880, WO08/86047 etc.), and it is commonly thought that these ROCK inhibitors have a therapeutic effect on hypertension, atherosclerosis, stroke, angina, arterial obstruction, peripheral arterial disease, peripheral circulation disorder, erectile dysfunction, acute and chronic pain, dementia, Alzheimer's disease, Parkinson's disease, neuronal degeneration, asthma, chronic obstructive pulmonary disease (COPD), pulmonary emphysema, chronic bronchitis, pulmonary fibrosis, interstitial pneumonia, pulmonary hypertension, amyotrophic lateral sclerosis, spinal cord injury, rheumatoid arthritis, osteoarthritis, osteoporosis, psoriasis, multiple sclerosis, diabetes, urinary organ disease such as overactive bladder (OAB) and benign prostatic hypertrophy (BPH), metastasis, cancer, glaucoma, ocular hypertension, retinopathy, auto immune disease, virus infection, myocardial protection etc.

Published Application No. WO93/08186 discloses compounds of the formula:

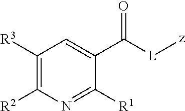

Wherein R¹ is C₁₋₆ alkoxy, C₃₋₈ cycloalkoxy or C₃₋₈ cylcoalkyl C₁₋₄ alkoxy; R² is hydrogen, halo, C₁₋₆ alkyl, C₁₋₆ alkoxy, or amino optionally substituted by one or two C₁₋₆ alkyl groups; R³ is hydrogen, halo or C₁₋₆ alkyl; L is O or NH and z is a di-azacyclic side chain. The compounds are useful as 5-HT₃ antagonists.

Published Application No. WO97/05129 discloses compound of the formula:

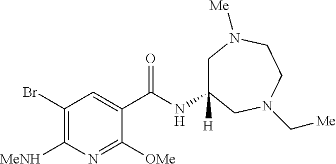

Which are useful as 5-HT₃ antagonists.

Published Application No. WO05/61442 discloses compound of the formula:

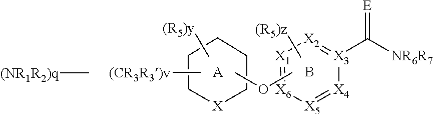

which are useful as opioid receptor antagonists.

Published Application No. WO03/82808 discloses compounds

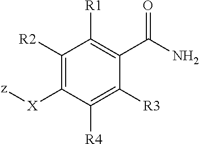

of the formula:
Which are useful as ROCK inhibitors.

### SUMMARY OF THE INVENTION

This invention relates to novel heterocyclic carboxamide derivatives and salts thereof.

More particularly, it relates to novel heterocyclic carboxamide derivatives and salts thereof which act as a ROCK inhibitor, to a pharmaceutical composition comprising the same and to a method of using the same therapeutically in the treatment and/or prevention of ROCK-related disease.

One object of this invention is to provide new and useful heterocyclic carboxamide derivatives and salts thereof which act as a ROCK inhibitor.

A further object of this invention is to provide a pharmaceutical composition comprising, as an active ingredient, said heterocyclic carboxamide derivatives and salts thereof.

Still further object of this invention is to provide a therapeutical method for the treatment and/or prevention of ROCK-related diseases in a patient in need of such disease, using said heterocyclic carboxamide derivatives and salts thereof.

The object heterocyclic carboxamide derivatives of this invention are new and can be represented by the following general formula [I] or its pharmaceutically acceptable salt:

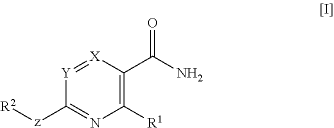

### Wherein

R¹ is hydrogen, halogen, optionally substituted lower alkyl, optionally substituted —O-lower alkyl, optionally substituted amino, or amino lower alkyl;

R² is cycloalkyl, heterocyclic group or lower alkyl each of which may be optionally substituted;

X and Y are each N or CR³ in which R³ is hydrogen, halogen, lower alkyl, —O-lower alkyl, trifluoromethyl, or amino;

z is bond, —O—, or —NR⁴—, in which R⁴ is hydrogen, or optionally substituted lower alkyl;

### DETAILED DESCRIPTION OF THE PRESENT INVENTION

In the above and subsequent description of the present specification, suitable examples of the various definitions to be included within the scope of the invention are explained in detail in the following.

Each of the terms “halogen”, “halo” and “Hal” may include fluorine, chlorine, bromine and iodine.

The term “lower” used in the description is intended to mean 1 to 6 carbon atom(s) unless otherwise indicated.

The “lower alkyl” used in the compound of the present invention may include straight-chain or branched-chain alkyl having 1 to 6 carbon atoms, such as methyl, ethyl, propyl, isopropyl, butyl, isobutyl, sec-butyl, tert-butyl, pentyl, isopentyl, neopentyl, tert-pentyl, 1-methylbutyl, 2-methylbutyl, 1,2-dimethylpropyl, hexyl, isohexyl, 1-methylpentyl, 2-methylpentyl, 3-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl and the like. Among them, those having 1 to 4 carbon atoms are preferred, and methyl, ethyl, propyl, isopropyl and tert-butyl are particularly preferred.

The “cycloalkyl” used in the compound of the present invention may include 3 to 8-membered saturated hydrocarbon group such as cyclopropyl, cyclobutyl, cyclopentyl, cyclohexyl, cycloheptyl and the like. Among them, cyclohexyl is more preferred.

The “heterocyclic group” used in the compound of the present invention may include 5 to 8-membered monocyclic group containing 1 to 4 hetero atoms selected from the group consisting of oxygen, nitrogen and sulfur atom, and bicyclic heterocyclic group in which 5 to 6-membered monocyclic heterocyclic ring is fused with benzene ring, cycloalkane ring or other monocyclic heterocyclic ring, and example of them includes heteroaryl group such as pyrrolyl, imidazolyl, pyrazolyl, pyridyl, pyrazinyl, pyrimidinyl, pyridazinyl, triazolyl, thienyl, thiopyranyl, furyl, pyranyl, dioxolanyl, thiazolyl, isothiazolyl, thiadiazolyl, thiazinyl, oxazolyl, isoxazolyl, oxadiazolyl, furazanyl, dioxazolyl, oxazinyl, oxadiazinyl, dioxazinyl and the like, saturated heterocyclic group such as pyrrolidinyl, piperidinyl, piperazinyl, morpholinyl, thiomorpholinyl and the like, and fused heterocyclic group such as indolyl, isoindolyl, indazolyl, indolinyl, isoindolinyl, quinolyl, quinazolinyl, quinoxalinyl, isoquinolyl, 3,4-dihydro-isoquinolyl, tetrahydroisoquinolyl, octahydro-isoquinolyl, benzimidazolyl, benzothienyl, benzothiazolyl, benzofuranyl, benzofurazanyl, imidazopyridyl, imidazopyrazinyl, pyridopyridyl, phthalazinyl, naphthyridinyl, indolizinyl, purinyl, quinolizinyl, cinnolinyl, isochromanyl, chromanyl and the like. Preferred one is 5 to 6-membered monocyclic heterocyclic group, or bicyclic heterocyclic group in which 5 to 6-membered monocyclic heterocyclic ring is fused with benzene ring or cyclohexane ring, having 1 or 2 hetero atoms selected from nitrogen atom, oxygen atom and sulfur atom, such as pyrrolyl, pyridyl, furyl, indolyl, indolinyl, thienyl, thiazolyl, benzofuranyl, benzothiazolyl, benzothienyl, quinolyl, 3,4-dihydro-isoquinolyl, tetrahydroisoquinolyl, and octahydro-isoquinolyl.

The substituent(s) of the “substituted” group used in the compound of the present invention may be any substituent which is generally used in the art as a substituent for the group, and the “substituted” group may have one or more substituents which are same or different each other.

And the “substituent(s)” of the “substituted lower alkyl” may include amino;

The “substituent(s)” of the “substituted —O-lower alkyl” may include halogen or —OH;

The “substituent (s)” of the “substituted amino” may include lower alkyl;

The “substituted amino” of the “cycloalkyl which is substituted with optionally substituted amino” may include —NH-lower alkyl, —N(lower alkyl)₂, —NH—SO₂-lower alkyl or —NH-lower alkyl-SO₂-lower alkyl;

Suitable pharmaceutically acceptable salts of the object compound [I] are conventional non-toxic salts and include, for example, a salt with an acid addition salt such as a salt with; an inorganic acid addition salt (e.g., hydrochloride, hydrobromide, sulfate, hydrogensulfate, phosphate, etc.); an organic carboxylic or sulfonic acid addition salt (e.g., formate, acetate, trifluoroacetate, maleate, tartrate, citrate, fumarate, methanesulfonate, benzenesulfonate, toluenesulfonate, etc.); and a salt with a basic or acidic amino acid (e.g., arginine, aspartic acid, glutamic acid, etc.).

The preferred embodiment of the present invention is shown as follow.

In the compound having the formula [I],

R¹ is hydrogen, halogen, —O-lower alkyl which may be substituted with halogen or —OH, or amino which may be substituted with lower alkyl;

R² is cycloalkyl which is substituted with optionally substituted amino, heterocyclic group, or amino lower alkyl;

X is CH or N;

Y is N or CR³ in which R³ is hydrogen, halogen or lower alkyl;

z is —O— or —NH—.

And more preferably,

R¹ is —O-lower alkyl which may be substituted with halogen or —OH;

R² is cycloalkyl which is substituted with —NH₂, —NH-lower alkyl, —N(lower alkyl)₂, —NH—SO₂-lower alkyl or —NH-lower alkyl-SO₂-lower alkyl;

X is CH or N;

Y is N or CR³ in which R³ is hydrogen, halogen or lower alkyl;

z is —NH—.

And most preferably, the compound having the formula [I], which is 6-[(trans-4-aminocyclohexyl)amino]-5-fluoro-2-methoxynicotinamide.

The compound of the present invention or a salt thereof can be prepared by those in the art referring the present specification and general methods known to those skilled in the art. Representative reaction processes used for synthesizing the compound of the present invention are shown as follow, but the reaction process used for synthesizing the compound of the present invention is not limited to the following exemplary processes.

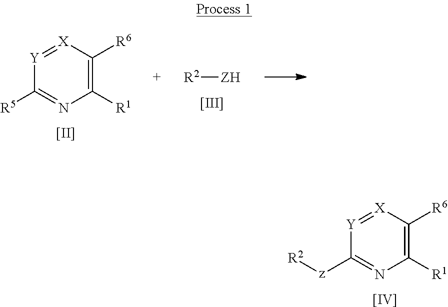

In the above formula, each of R¹, R², X, Y and z represents the same meaning as defined above, and R⁵ is a leaving group, such as chloro, fluoro, trifluoromethanesulfonyloxy, and R⁶ is cyano and methoxycarbonyl. Process 1 is the process for preparing Compound [IV], wherein Compound [IV] is synthesized by substitution reaction of Compound [II] with Compound [III].

Compound [II] and [III] may be purchased if it is commercial, or synthesized according to general methods obvious to the person skilled in the organic chemistry from commercial compounds.

In the case, base is generally used. The base employable in this process is not particularly limited so long as it accelerates this process and may include organic amines such as triethylamine, tributylamine, diisopropylethylamine (DIEA).

The solvent employable in this process is not particularly limited so long as it is inactive in this reaction and may include amides such as N,N-dimethylformamide, N,N-dimethylacetamide and 1,3-dimethylimidazolidinone. The temperature at that time varies depending on the starting material, the solvent, or the like, but it is usually from ambient temperature to 150° C.

The reaction time after the adding base varies depending on the starting material, the solvent, or the like, but it is usually from 1 hr to 30 hrs.

After the reaction, the mixture is partitioned between water and organic solvent insoluble with water such as ethyl acetate, chloroform, or the like, and organic layer is separated. The organic layer is washed by water, hydrochloric acid, saturated sodium hydrogencarbonate solution, brine, or the like, dried over anhydrous magnesium sulfate or sodium sulfate, and evaporated in vacuo. The target compound is purified by the conventional method such as silica gel column chromatography, or the like.

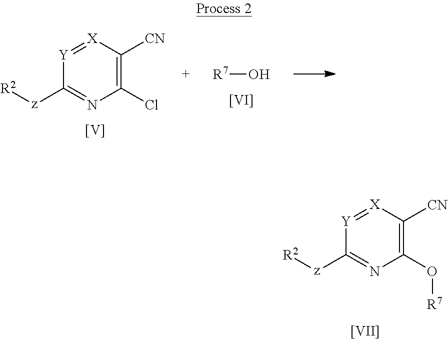

In the above formula, each of R², X, Y and z represents the same meaning as defined above, and R⁷ is optionally substituted alkyl. Process 2 is the process for preparing Compound [VII], wherein Compound [VII] is synthesized by substitution reaction of Compound [V] with alcohol [VI].

Compound [V] and [VI] may be purchased if it is commercial, or synthesized according to general methods obvious to the person skilled in the organic chemistry from commercial compounds.

In this process, the Compound [V] is added to the mixture of Compound [VI] and base in the solvent, and the mixture is stirred at ambient temperature to 150° C.

The solvent employable in dissolution of Compound [V] and [VI] is not particularly limited so long as it is inactive, and may include tetrahydrofuran, dioxane, N,N-dimethylformamide, etc. The preferred base is potassium tert-butoxide, cesium carbonate, and sodium hydroxide etc.

The reaction time varies depending on the starting material, the solvent, or the like, but it is usually from 1 hr to 30 hrs.

After the reaction, the mixture is partitioned between water and organic solvent insoluble with water such as ethyl acetate, chloroform, or the like, and organic layer is separated. The organic layer is washed by water, hydrochloric acid, saturated sodium hydrogencarbonate solution, brine, or the like, dried over anhydrous magnesium sulfate or sodium sulfate, and evaporated in vacuo. The target compound is purified by the conventional method such as silica gel column chromatography, or the like.

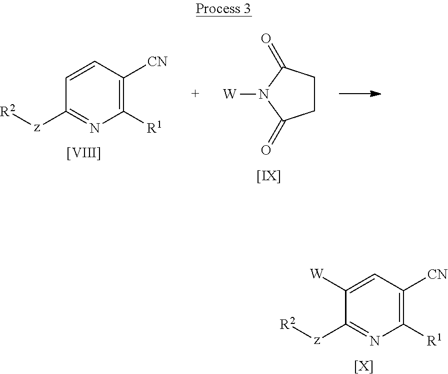

In the above formula, each of R¹, R² and z represents the same meaning as defined above, and W is chlorine, bromine or iodine. Process 3 is the process for preparing Compound [X], wherein Compound [X] is synthesized by halogenation of Compound [VIII] with Compound [IX].

Compound [VIII] and [IX] may be purchased if it is commercial, or synthesized according to general methods obvious to the person skilled in the organic chemistry from commercial compounds.

In this process, first, Compound [VIII] is dissolved in solvent, and added Compound [IX] and stirred under elevated temperature e.g., about 70° C. to 90° C.

The solvent employable in dissolution of Compound [VIII] is not particularly limited so long as it is inactive, and may include 2-propanol, ethanol, N,N-dimethylformamide etc.

The reaction time varies depending on the starting material, the solvent, or the like, but it is usually from 1 hr to 30 hrs.

After the reaction, the mixture is partitioned between water and organic solvent insoluble with water such as ethyl acetate, chloroform, or the like, and organic layer is separated. The organic layer is washed by brine, or the like, dried over anhydrous magnesium sulfate or sodium sulfate, and evaporated in vacuo. The target compound is purified by the conventional method such as silica gel column chromatography, or the like.

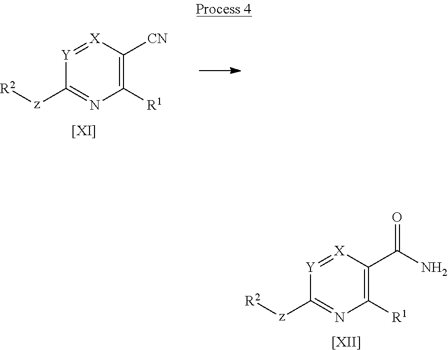

In the above formula, each of R¹, R², X, Y and z represents the same meaning as defined above. Process 4 is the process for preparing Compound [XII], wherein Compound [XII] is synthesized by hydrolysis of Compound [XI].

Compound [XI] may be purchased if it is commercial, or synthesized according to general methods obvious to the person skilled in the organic chemistry from commercial compounds.

Process 4 is the process for preparing amide Compound [XII] from nitrile Compound [XI], hydrogen peroxide and sodium hydroxide in solvent.

The solvent employable in this process is not particularly limited so long as it is inactive in this reaction and may include dimethylsulfoxide and ethanol. The temperature at that time varies depending on the starting material, the solvent, or the like, but it is usually room temperature.

The reaction time varies depending on the starting material, the solvent, or the like, but it is usually from 1 hr to 30 hrs.

After the reaction, the mixture is partitioned between water and organic solvent insoluble with water such as ethyl acetate, chloroform, or the like, and organic layer is separated. The organic layer is washed by water, hydrochloric acid, saturated sodium hydrogencarbonate solution, brine, or the like, dried over anhydrous magnesium sulfate or sodium sulfate, and evaporated in vacuo. The target compound is purified by the conventional method such as silica gel column chromatography, or the like.

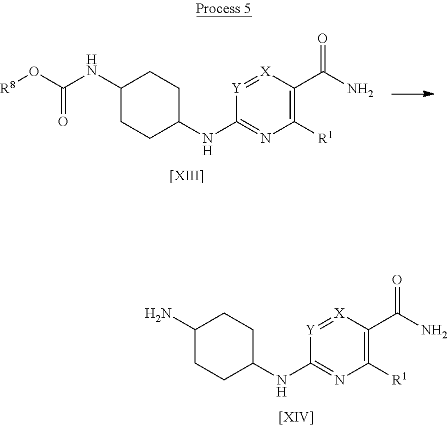

In the above formula, each of R¹, X and Y represents the same meaning as defined above, and R⁸ is tert-butyl or benzyl.

Process 5 is the process for preparing Compound [XIV], wherein Compound [XIV] is synthesized by decarbamation of Compound [XIII].

Compound [XIII] may be synthesized according to general methods obvious to the person skilled in the organic chemistry from commercial compounds.

Process 5 is the process for preparing the Compound [XIV] from Compound [XIII] by acidic deprotection (R⁸ is tert-butyl) or hydrogeneation (R⁸ is benzyl) in solvent.

In the case of tert-butyl, acid is generally used. The acid employable in this process is not particularly limited so, long as it accelerates this process and may include, for example, hydrogen chloride and trifluoroacetic acid. The solvent employable in this process is not particularly limited so long as it is inactive in this reaction and may include dichloromethane, methanol, 1,4-dioxane, chloroform etc.

In the case of benzyl, hydrogenation is generally used. The catalyst employable in this process is not particularly limited so long as it accelerates this process and may include, for example, palladium on charcoal and palladium hydroxide on carbon. The solvent employable in this process is not particularly limited so long as it is inactive in this reaction and may include dichloromethane, chloroform, methanol, 1,4-dioxane, tetrahydrofuran etc.

The temperature at that time varies depending on the starting material, the solvent, or the like, but it is preferably room temperature.

The reaction time varies depending on the starting material, the solvent, or the like, but it is usually from 1 hr to 30 hrs.

After the reaction, the mixture is concentrated in vacuo, and the target compound is purified by the conventional method such as silica gel column chromatography, or the like.

The compounds obtained by the above processes can be isolated and purified by a conventional method such as pulverization, recrystallization, column chromatography, reprecipitation, or the like, and converted to the desired salt in conventional manners, if necessary.

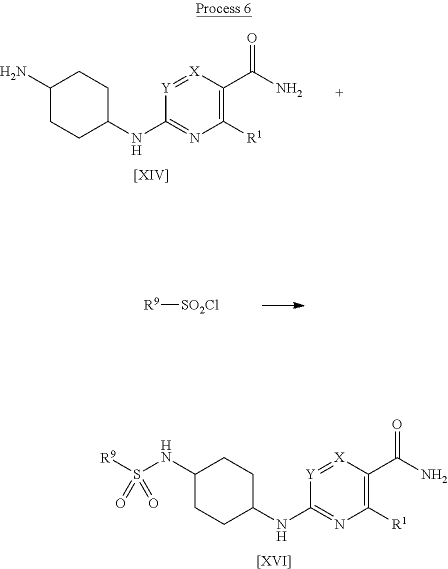

In the above formula, each of R¹, X and Y represents the same meaning as defined above, and R⁹ is optionally substituted alkyl.

Process 6 is the process for preparing Compound [XVI], wherein Compound [XVI] is synthesized by sulfonylation of Compound [XIV].

Compound [XIV] can be synthesized by Process 5.

Process 6 is the process for preparing Compound [XVI] from Compound [XIV], sulfonyl chloride and base in solvent.

The solvent employable in this process is not particularly limited so long as it is inactive in this reaction and may include dichloromethane etc. The temperature at that time varies depending on the starting material, the solvent, or the like, but it is usually room temperature.

The reaction time varies depending on the starting material, the solvent, or the like, but it is usually from 1 hr to 30 hrs.

After the reaction, the mixture is partitioned between water and organic solvent insoluble with water such as ethyl acetate, chloroform, or the like, and organic layer is separated. The organic layer is washed by water, hydrochloric acid, saturated sodium hydrogencarbonate solution, brine, or the like, dried over anhydrous magnesium sulfate or sodium sulfate, and evaporated in vacuo. The target compound is purified by the conventional method such as silica gel column chromatography, or the like.

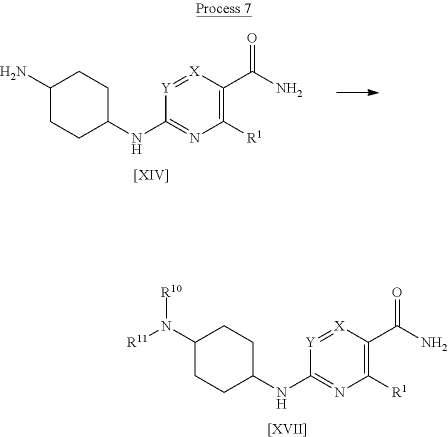

In the above formula, each of R¹, X and Y represents the same meaning as defined above, and R¹⁰ and is optionally substituted alkyl.

Process 7 is the process for preparing Compound [XVII], wherein Compound [XVII] is synthesized by reductive amination of Compound [XIV].

Compound [XIV] can be synthesized by Process 5.

Process 7 is the process for preparing Compound [XVII] from Compound [XIV], aldehyde and reducing agent in solvent.

The solvent employable in this process is not particularly limited so long as it is inactive in this reaction and may include dichloromethane etc. The temperature at that time varies depending on the starting material, the solvent, or the like, but it is usually room temperature.

The reaction time varies depending on the starting material, the solvent, or the like, but it is usually from 1 hr to 30 hrs.

After the reaction, the mixture is concentrated in vacuo, and the target compound is purified by the conventional method such as silica gel column chromatography, or the like.

It is to be noted that the compound [I] may include one or more stereoisomers due to asymmetric carbon atoms, and all of such isomers and mixture thereof are included within the scope of this invention.

It is also to be noted that the compound [I] may be a salt. For example, when a basic group such as an amino group is present in a molecule, the salt is exemplified by an acid addition salt (e.g. salt with an inorganic acid such as hydrochloric acid, hydrobromic acid, sulfuric acid, etc., salt with an organic acid such as methanesulfonic acid, benzenesulfonic acid, 4-toluenesulfonic acid, camphorsulfonic acid (e.g., [(1S,4R)-7,7-dimethyl-2-oxobicyclo[2.2.1]hept-1-yl]methanesulfonic acid or an enantiomer thereof, etc.), fumaric acid, maleic acid, mandelic acid, citric acid, salicylic acid, malonic acid, glutaric acid, succinic acid, etc.), etc., and when an acidic group such as carboxyl group is present, the salt is exemplified by a basic salt (e.g. salt with a metal such as lithium, sodium, potassium, calcium, magnesium, aluminium, etc., a salt with amino acid such as lysine, etc.), etc.

It is also to be noted that the solvating form of the compound [I] (e.g. hydrate, etc.) and any form of the crystal of the compound [I] are included within the scope of the present invention.

It is also to be noted that pharmaceutically acceptable prodrugs of the compound [I] are included within the scope of the present invention. Pharmaceutically acceptable prodrug means compound having functional groups which can be converted to —COOH, —NH₂, —OH etc in physiological condition to form the compound [I] of the present invention.

The compounds of the present invention or a salt thereof can inhibit an activity of any Rho kinase such as ROCK I and ROCK II. Therefore, the compounds of the present invention are useful for the treatment and/or prevention of a variety of ROCK-related diseases. ROCK-related disease which can be treated and/or prevented by using the compound of the present invention includes, but is not limited to, hypertension, atherosclerosis, stroke, angina, arterial obstruction, peripheral arterial disease, peripheral circulation disorder, erectile dysfunction, acute and chronic pain, dementia, Alzheimer's disease, Parkinson's disease, neuronal degeneration, asthma, chronic obstructive pulmonary disease, idiopathic pulmonary fibrosis, interstitial pulmonary fibrosis, amyotrophic lateral sclerosis, spinal cord injury, rheumatoid arthritis, osteoarthritis, osteoporosis, psoriasis, multiple sclerosis, diabetes, urinary organ diseases such as overactive bladder (OAB) and benign prostatic hypertrophy (BPH), metastasis, cancer, glaucoma, ocular hypertension, retinopathy, auto immune disease, virus infection, myocardial protection. Because the compounds of the present invention or a salt thereof have pain alleviation, cartilage protection effect and so on, preferably, ROCK-related disease which can be treated and/or prevented by using the compound of the present invention are osteoarthritis, peripheral arterial disease, benign prostatic hypertrophy (BPH), idiopathic pulmonary fibrosis, glaucoma or ocular hypertension.

For therapeutic purpose, the compound [I] and a pharmaceutically acceptable salt thereof of the present invention can be used in a form of pharmaceutical preparation containing one of said compounds, as an active ingredient, in admixture with a pharmaceutically acceptable carrier such as an organic or inorganic solid or liquid excipient suitable for oral, parenteral, external including topical, internal, intravenous, intramuscular, inhalant, nasal, intraarticular, intraspinal, transtracheal or transocular administration. The pharmaceutical preparations may be solid, semi-solid or solutions such as capsules, tablets, pellets, dragees, powders, granules, suppositories, ointments, creams, lotions, inhalants, injections, cataplasms, gels, tapes, eye drops, solution, syrups, aerosols, suspension, emulsion, or the like. If desired, there may be included in these preparations, auxiliary substances, stabilizing agents, wetting or emulsifying agents, buffers and other commonly used additives.

While the dosage of the compound [I] will vary depending upon the age and condition of a patient, an average single dose of about 0.1 mg, 1 mg, 10 mg, 50 mg, 100 mg, 250 mg, 500 mg and 1000 mg of the compound [I] may be effective for treating ROCK-related diseases. In general, amounts between 0.1 mg/body and about 1,000 mg/body may be administered per day.

The following Preparations and Examples are given for the purpose of illustrating this invention.

### EXAMPLE

### Preparation 1

A solution of methyl 2,6-difluoronicotinate (7.80 g) in N,N-dimethylformamide (100 mL) was added over 20 minutes at ambient temperature to a mixture of diisopropylethylamine (14.6 g) and tert-butyl (trans-4-aminocyclohexyl)carbamate (14.5 g) in N,N-dimethylformamide (134 mL) and the mixture was stirred at ambient temperature for overnight. The mixture was poured into water (800 mL) and the resulting precipitates were collected by filtration and washed with water. The precipitates were dissolved into ethyl acetate and dried over magnesium sulfate and evaporated in vacuo. The residue was purified by column chromatography on silica gel (toluene:Ethyl acetate=4:1-3:1) and triturated with toluene to afford methyl 6-({trans-4-[(tert-butoxycarbonyl)amino]cyclohexyl}amino)-2-fluoronicotinate (7.90 g) as a pale-yellow solid.

1H-NMR (DMSO-d6): δ 1.26-1.31 (4H, m), 1.45 (9H, s), 2.05-2.10 (4H, m), 2.65 (1H, m), 2.72 (1H, m), 3.86 (3H, d, J=2.7 Hz), 4.42 (1H, m), 4.50 (1H, m), 6.18 (1H, dd, J=1.9, 8.5 Hz), 8.03 (1H, t, J=8.5 Hz)

MS (ESI, m/z): 390 (M+Na)

### Preparation 2

To a solution of potassium tert-butoxide (46 mg) in absolute methanol (4 mL) was added methyl 6-({trans-4-[(tert-butoxycarbonyl)amino]cyclohexyl}amino)-2-fluoronicotinate (100 mg) and the mixture was stirred at 50° C. for 5 hours. The mixture was poured into dilute hydrochloric acid and the resultant precipitates were collected by filtration, washed with water and dried in vacuo to afford methyl 6-({trans-4-[(tert-butoxycarbonyl)amino]cyclohexyl}amino)-2-methoxynicotinate (100 mg) as a powder.

1H-NMR (DMSO-d6): δ 1.2-1.3 (4H, m), 1.38 (9H, s), 1.7-2.0 (4H, M), 3.15-3.25 (1H, m), 3.5-3.6 (1H, m), 3.66 (3H, s), 3.74 (3H, s), 6.08 (1H, d, J=8.6 Hz), 6.37 (1H, d, J=8.4 Hz), 0.35 (1H, m), 7.7-7.8 (1H, m), 7.88 (1H, d, J=8.4 Hz).

MS (ESI, m/z): 402 (M+Na)

### Preparation 3

To a solution of methyl 6-({trans-4-[(tert-butoxycarbonyl)amino]cyclohexyl}amino)-2-methoxynicotinate (680 mg) in methanol (15 mL) and tetrahydrofuran (15 mL) was added 1N sodium hydroxide aqueous solution and the mixture was stirred at 60° C. for 3 hours. After cooling to ambient temperature, the mixture was neutralized by addition of 1N hydrochloric acid and the resulting precipitates were collected by filtration, washed water and dried in vacuo to afford 6-({trans-4-[(tert-butoxycarbonyl)amino]cyclohexyl}amino)-2-methoxynicotinic acid (600 mg) as a powder.

1H-NMR (DMSO-d6): δ 1.15-1.3 (4H, m), 1.38 (9H, s), 1.7-2.0 (4H, m), 3.15-3.25 (1H, m), 3.82 (3H, s), 6.02 (1H, d, J=8.5 Hz), 6.74 (1H, d, J=7.6 Hz), 7.15-7.25 (1H, m), 7.76 (1H, d, J=8.5 Hz).

MS (negative ESI, m/z): 364 (M−H)

### Preparation 4

To a solution of 6-{(trans-4-[(tert-butoxycarbonyl)amino]cyclohexyl}amino)-2-methoxynicotinic acid (600 mg) in N,N-dimethylformamide (6 mL) were added N-[3-(Dimethylamino) propyl]-N′-ethylcarbodiimide hydrochloride (393 mg) and 1H-1,2,3-benzotriazol-1-ol (277 mg) and the mixture was stirred at ambient temperature for 1.5 hours. Then aqueous ammonia solution was added and the reaction mixture was stirred at ambient temperature. for 1 hour. The mixture was neutralized with 1N hydrochloric acid, the resulting precipitates were collected, and washed by water. The precipitates were purified by preparative TLC developing with a solution of chloroform:methanol (30:1) to afford tert-butyl {trans-4-[(5-carbamoyl-6-methoxypyridin-2-yl)amino]cyclohexyl}carbamate (160 mg) as a powder.

1H-NMR (CDCl₃): δ 1.20-1.39 (4H, m), 1.45 (9H, s), 2.08-2.17 (4H, m), 3.46 (1H, br s), 3.60 (1H, br s), 4.00 (3H, s), 4.42 (1H, br s), 5.72 (1H, br s), 6.03 (1H, d, J=8.5 Hz), 7.52 (1H, d, J=2.8 Hz), 8.20 (1H, d, J=8.5 Hz)

MS (ESI, m/z): 387 (M+Na)

### Example 1

To a solution of tert-butyl {trans-4-[(5-carbamoyl-6-methoxypyridin-2-yl)amino]cyclohexyl}carbamate (160 mg) in chloroform/methanol (10:1) (3 ml) was added 4N hydrogen chloride in ethyl acetate, and stirred overnight. The resulting precipitates, were collected by filtration and washed with ethyl acetate, and dried in vacuo to afford 6-[(trans-4-aminocyclohexyl)amino]-2-methoxynicotinamide dihydrochloride (147 mg) as a powder.

1H-NMR (DMSO-d6): δ 1.90-1.50 (4H, m), 1.96-2.06 (4H, m), 3.00 (1H, br s), 3.64 (1H, br s), 3.91 (3H, s), 6.11 (1H, d, J=8.5 Hz), 7.20 (2H, br s), 7.87 (1H, d, J=8.5 Hz), 8.12 (4H, m)

MS (ESI, m/z): 265 (M+H)

### Preparation 5

To a solution of 2,4-chloronicotinonitrile (3.0 g) and tert-butyl (trans-4-aminocyclohexyl)carbamate) (4.39 g) in N,N-dimethylformamide (32 mL) were added diisopropylethylamine (4.48 g, 34.7 mmol) and the mixture was stirred at 90° C. for 2 hours. The mixture was poured into ice-water (150 mL) and the resulting precipitates were collected by filtration, washed with diisopropylether and dried in vacuo. The crude product was dissolved in chloroform/methanol (10:1) (10 mL) and purified by column chromatography on silica gel eluting with toluene:ethyl acetate (4:1) and crystallized from toluene to afford tert-butyl {trans-4-[(6-chloro-5-cyanopyridin-2-yl)amino]cyclohexyl}carbamate (4.5 g) as crystals.

1H-NMR (DMSO-d6): δ 1.20-1.30 (4H, m), 1.38 (9H, s), 1.79 (2H, br s), 1.89 (2H, br s), 3.23 (1H, br s), 3.65 (1H, br s), 6.47 (1H, d, J=8.16 Hz), 6.76 (1H, d, J=7.72 Hz), 7.70 (1H, d, J=8.16 Hz), 7.96 (1H, d, J=7.52 Hz)

MS (ESI, m/z): 373 (M+Na)

### Preparation 6

The following compound was obtained in a similar manner to that of Preparation 2:

### tert-butyl {trans-4-[(5-cyano-6-methoxypyridin-2-yl)amino]cyclohexyl}carbamate

1H-NMR (DMSO-d6): δ 1.20-1.38 (4H, m), 1.45 (9H, s), 2.00-2.15 (4H, m), 3.46 (1H, br s), 3.61 (1H, br s), 3.93 (3H, s), 4.40 (1H, br s), 4.85 (1H, br s), 5.91 (1H, d, J=8.44 Hz), 7.48 (1H, d, J=8.44 Hz)

MS (ESI, m/z): 369 (M+Na)

### Preparation 7

To a cooled solution of tert-butyl {trans-4-[(5-cyano-6-methoxypyridin-2-yl)amino]cyclohexyl}carbamate (620 mg) in dimethylsulfoxide (24 mL) was added 1N sodium hydroxide (2.7 mL), followed by dropwise addition of 30% hydrogen peroxide (4.0 mL) and the mixture was stirred at ambient temperature for 24 hours. The mixture was poured into water (100 mL) and the resulting precipitates were collected by filtration, washed with water and dried in vacuo to afford tert-butyl {trans-4-[(5-carbamoyl-6-methoxypyridin-2-yl)amino]cyclohexyl}carbamate (590 mg) as a powder.

1H-NMR (DMSO-d6): δ 1.21-1.38 (4H, m), 1.45 (9H, s), 2.07-2.16 (4H, m), 3.46 (1H, br s), 3.60 (1H, br s), 4.00 (3H, s), 4.42 (1H, br s), 4.80 (1H, br m), 5.71 (1H, br s), 6.03 (1H, d, J=8.6 Hz), 7.51 (1H, br s), 8.20 (1H, d, J=8.5 Hz)

MS (ESI, m/z): 365 (M+H)

### Preparation 8

The product of Preparation 6 was synthesized by another route. To a solution of 6-hydroxy-2-methoxynicotinonitrile (1.0 g) in dichloromethane (30 ml) was added triethylamine (1.09 mL), followed by dropwise addition of a solution of trifluoromethanesulfonic anhydride (2.07 g) in dichloromethane (10 mL) at 5° C. After stirring at 5° C. for 1 hour, water (20 mL) was added and the mixture was extracted with ethyl acetate. The organic layer was washed with 1N hydrochloric acid and brine, dried over magnesium sulfate, and evaporated in vacuo to afford crude 5-cyano-6-methoxypyridin-2-yl trifluoromethanesulfonate (1.4 g) as a blackish brown oil. The crude oil was dissolved in 1,3-dimethylimidazolidinone (15 mL) were added tert-butyl (trans-4-aminocyclohexyl)carbamate (1.17 g) and diisopropylethylamine (0.96 g) and the mixture was stirred at 90° C. for 2 hours. The mixture was poured into water and extracted with ethyl acetate. The organic layer was washed with water and brine, dried over magnesium sulfate, and evaporated in vacuo. The residue was purified by column chromatography on silica gel eluting with hexane:ethyl acetate (4:6) to afford tert-butyl {trans-4-[(5-cyano-6-methoxypyridin-2-yl)amino]cyclohexyl}carbamate (840 mg) as a yellow powder.

1H-NMR (DMSO-d6): δ 1.20-1.38 (4H, m), 1.45 (9H, s), 2.00-2.15 (4H, m), 3.46 (1H, br s), 3.61 (1H, br s), 3.93 (3H, s), 4.40 (1H, br s), 4.85 (1H, br s), 5.91 (1H, d, J=8.44 Hz), 7.48 (1H, d, J=8.44 Hz)

MS (ESI, m/z): 369 (M+Na)

### Preparation 9

The following compound was obtained in a similar manner to that of Preparation 1:

### tert-butyl {trans-4-[(6-chloro-5-cyano-3-fluoropyridin-2-yl)amino]cyclohexyl}carbamate

1H-NMR (DMSO-d6): δ 1.15-1.3 (2H, m), 1.38 (9H, s), 1.35-1.5 (2H, m), 1.75-1.9 (4H, m), 3.15-3.25 (1H, m), 3.75-3.85 (1H, m), 6.76 (1H, d, J=8.0 Hz), 7.94 (1H, d, J=10.7 Hz), 8.02 (1H, d, J=8.0 Hz)

MS (ESI, m/z): 369 (M+H)

### Preparation 10

The following compound was obtained in a similar manner to that of Preparation 2:

### tert-butyl {trans-4-[(5-cyano-3-fluoro-6-methoxypyridin-2-yl)amino]cyclohexyl}carbamate

1H-NMR (CDCl₃): δ 1.32 (3H, s), 1.23-1.41 (4H, m), 1.45 (9H, s), 2.10-2.18 (4H, m), 3.47 (1H, m), 3.85-3.92 (1H, m), 3.93 (3H, s), 4.40 (1H, br s), 4.91 (1H, m), 7.23 (1H, d, J=6.8 Hz)

MS (ESI, m/z): 387 (M+Na)

### Preparation 11

The following compound was obtained in a similar manner to that of Preparation 7:

### tert-butyl{trans-4-[(5-carbamoyl-3-fluoro-6-methoxypyridin-2-yl)amino]cyclohexyl}carbamate

1H-NMR (CDCl₃): δ 1.23-1.42 (4H, m), 1.45 (9H, s), 2.10-2.20 (4H, m), 3.50 (1H, br s), 3.85-3.93 (1H, m), 4.00 (3H, s), 4.41 (1H, s), 4.82 (1H, br s), 5.85 (1H, br s), 7.59 (1H, br s), 7.93 (1H, d, J=11.3 Hz)

MS (ESI, m/z): 405 (M+Na)

### Example 2

The following compound was obtained in a similar manner to that of Example 1:

### 6-[(trans-4-aminocyclohexyl)amino]-5-fluoro-2-methoxynicotinamide dihydrochloride

1H-NMR (DMSO-d6): δ 1.39-1.51 (4H, m), 1.91-2.01 (4H, m), 2.97 (1H, s), 3.80 (1H, br s), 3.95 (3H, s), 7.20 (1H, br s), 7.36 (2H, br s), 7.70 (1H, d, J=11.6 Hz), 8.17 (4H, m)

MS (ESI, m/z): 283 (M+H)

### Preparation 12

The following compound was obtained in a similar manner to that of Preparation 1:

### benzyl {trans-4-[(6-chloro-5-cyano-3-fluoropyridin-2-yl)amino]cyclohexyl}carbamate

1H-NMR (DMSO-d6): δ1.20-1.34 (2H, m), 1.36-1.50 (2H, m), 1.75-1.91 (4H, m), 3.21-3.34 (1H, m), 3.74-3.86 (1H, m), 5.00 (2H, s), 7.24 (1H, d, J=7.9 Hz), 7.28-7.40 (5H, m), 7.94 (1H, d, J=10.7 Hz), 8.02 (1H, d, J=7.6 Hz).

MS (ESI, m/z): 425 (M+Na)

### Preparation 13

To a solution of benzyl {trans-4-[(6-chloro-5-cyano-3-fluoropyridin-2-yl)amino]cyclohexyl}carbamate (83.3 g) in methanol (1250 mL) and tetrahydrofuran (278 mL) was added cesium carbonate (538.8 g) and the mixture was refluxed for 3.5 hours. The solution was cooled to ambient temperature and poured into 10% sodium chloride aqueous solution (7 L) and the resulting precipitates were collected by filtration, washed with water and dried in vacuo. The powder was dissolved in chloroform/methanol (9:1) (500 mL) and the insoluble materials were filtered off. To the solution was added silica gel (400 g) and evaporated in vacuo. The crude product was purified by column chromatography on silica gel eluting with hexane:ethyl acetate (2:1) to afford benzyl {trans-4-[(5-cyano-3-fluoro-6-methoxypyridin-2-yl)amino]cyclohexyl}carbamate (31.36 g) as a pale-yellow powder.

1H-NMR (DMSO-d6): δ1.22-1.50 (4H, m), 1.81-1.96 (4H, m), 3.21-3.34 (1H, m), 3.76-3.88 (1H, m), 3.87 (3H, s), 5.00 (2H, s), 7.22 (1H, d, J=7.8 Hz), 7.28-7.40 (5H, m), 7.61 (1H, d, J=7.3 Hz), 7.72 (1H, d, J=10.7 Hz).

MS (ESI, m/z): 421 (M+Na)

### Preparation 14

The following compound was obtained in a similar manner to that of Preparation 7:

### benzyl {trans-4-[(5-carbamoyl-3-fluoro-6-methoxypyridin-2-yl)amino]cyclohexyl}carbamate

1H-NMR (DMSO-d6): δ 1.22-1.48 (4H, m), 1.82-2.00 (4H, m), 3.23-3.34 (1H, m), 3.74-3.86 (1H, m), 3.91 (3H, s), 5.01 (2H, s), 7.12 (1H, d, J=7.8 Hz), 7.22 (1H, d, J=7.5 Hz), 7.28-7.40 (7H, m), 7.69 (1H, d, J=11.6 Hz).

MS (ESI, m/z): 439 (M+Na)

### Example 3

To a solution of benzyl {trans-4-[(5-carbamoyl-3-fluoro-6-methoxypyridin-2-yl)amino]cyclohexyl}carbamate (22.17 g) in ethyl acetate (443 mL) and methanol (443 mL) was added 10% Palladium on charcoal (50% wet) (2.22 g) and the mixture was hydrogenated under atmospheric pressure for 1.5 hours. The catalyst was filtered off and the filtrate was evaporated in vacuo. The residue was purified by column chromatography on NH silica gel (Fuji Silysia chemical Ltd.) eluting with chloroform, and recrystallized twice with ethyl acetate to afford 6-[(trans-4-aminocyclohexyl)amino]-5-fluoro-2-methoxynicotinamide (4.76 g) as pale-yellow crystals.

1H-NMR (CDCl₃): δ 1.00-1.19 (2H, m), 1.31-1.55 (3H, m), 1.73-1.95 (4H, m), 2.48-2.56 (1H, m), 3.76-3.84 (1H, m), 3.91 (3H, s), 7.08 (1H, d, J=7.2 Hz), 7.31 (1H, bs), 7.36 (1H, bs), 7.68 (1H, d, J=11.8 Hz).

MS (ESI, m/z): 283 (M+H)

### Preparation 15

A solution of 2,6-dichloro-5-fluoronicotinonitrile (5.0 g) and trans-cyclohexane-1,4-diamine (5.98 g) in N,N-dimethylformamide (50 mL) was stirred at ambient temperature for 2 hours. The mixture was poured into a mixture of water (400 mL) and dichloromethane (400 mL) and the insoluble materials were filtered off. The organic layer was washed with brine, dried over sodium sulfate, and evaporated in vacuo. The residue was triturated with ethyl acetate to afford 6-[(trans-4-aminocyclohexyl)amino]-2-chloro-5-fluoronicotino nitrile (3.83 g) as a powder.

1H-NMR (DMSO-d6): δ1.04-1.16 (2H, m), 1.32-1.56 (3H, m), 1.74-1.84 (4H, m), 2.45-2.54 (1H, m), 3.74-3.86 (1H, m), 7.92 (1H, d, J=10.8 Hz), 7.97 (1H, bs).

MS (ESI, m/z): 269 (M+H)

### Preparation 16

The following compound was obtained in a similar manner to that of Preparation 13:

### 6-[(trans-4-aminocyclohexyl)amino]-5-fluoro-2-methoxynicotinonitrile

1H-NMR (DMSO-d6): δ1.05-1.18 (2H, m), 1.32-1.50 (3H, m), 1.74-1.91 (4H, m), 2.47-2.56 (1H, m), 3.76-3.88 (1H, m), 3.87 (3H, s), 7.57 (1H, d, J=7.4 Hz), 7.70 (1H, d, J=10.9 Hz).

MS (ESI, m/z): 265 (M+H)

### Preparation 17

To a solution of 6-[(trans-4-aminocyclohexyl)amino]-5-fluoro-2-methoxynicotinonitrile (2.81 g) in dichloromethane was added diisopropylethylamine (2.06 g), followed by dropwise addition of benzyl chloroformate (2.18 g) and the mixture was stirred at ambient temperature for 30 minutes. The mixture was poured into water and extracted with ethyl acetate. The organic layer was washed with saturated sodium bicarbonate aqueous solution, water and brine, dried over sodium sulfate and evaporated in vacuo. The residue was triturated with diisopropylether and recrystallized from 2-propanol to afford benzyl {trans-4-[(5-cyano-3-fluoro-6-methoxypyridin-2-yl)amino]cyclohexyl}carbamate (3.08 g).

1H-NMR (DMSO-d6): δ1.22-1.50 (4H, m), 1.82-1.96 (4H, m), 3.22-3.36 (1H, m), 3.76-3.91 (1H, m), 3.87 (3H, s), 5.00 (2H, s), 7.22 (1H, d, J=7.8 Hz), 7.28-7.39 (5H, m), 7.61 (1H, d, J=7.3 Hz), 7.72 (1H, d, J=10.8 Hz).

MS (ESI, m/z): 421 (M+Na)

### Preparation 18

To a solution of tert-butyl {trans-4-[(5-cyano-6-methoxypyridin-2-yl)amino]cyclohexyl}carbamate (1.4 g) in 2-propanol (28 mL) was added N-chlorosuccinimide (0.65 g) and the mixture was stirred at 80° C. for 2 hours. The mixture was evaporated in vacuo and the residue was purified by column chromatography on silica gel eluting with chloroform/hexane (4:1) to afford tert-butyl {trans-4-[(3-chloro-5-cyano-6-methoxypyridin-2-yl)amino]cyclohexyl}carbamate (1.34 g) as a white powder.

1H-NMR. (DMSO-d6): δ 1.15-1.25 (2H, m), 1.4-1.55 (2H, m), 1.75-1.95 (4H, m), 3.1-3.25 (1H, m). 3.8-3.9 (1H, m), 3.87 (3H, s), 6.75 (1H, d, J=7.9 Hz), 7.14 (1H, d, J=7.7 Hz), 7.93 (1H, s).

MS (ESI, m/z): 405, 403 (M+Na)

### Preparation 19

The following compound was obtained in a similar manner to that of Preparation 7:

### tert-butyl {trans-4-[(5-carbamoyl-3-chloro-6-methoxypyridin-2-yl)amino]cyclohexyl}carbamate

1H-NMR (DMSO-d6): δ 1.18-1.56 (13H, m), 1.76-1.98 (4H, m), 3.14-3.27 (1H, m), 3.77-3.89 (1H, m), 3.93 (3H, s), 6.68 (1H, d, J=7.8 Hz), 6.75 (1H, d, J=7.8 Hz), 7.29-7.41 (2H, m), 7.93 (1H, s)

MS (ESI, m/z): 421 (M+Na)

### Example 4

To a solution of tert-butyl {trans-4-[(5-carbamoyl-3-chloro-6-methoxypyridin-2-yl)amino]cyclohexyl}carbamate (1.25 g) in dichloromethane (25 mL) was added trifluoroacetic acid (3.57 g) and the mixture was stirred at ambient temperature for 18 hours. The mixture was evaporated in vacuo and the residue was purified by column chromatography on NH silica gel (Fuji Silysia Chemicals Ltd.) eluting with chloroform/methanol (10:1) to afford (6-[(trans-4-aminocyclohexyl)amino]-5-chloro-2-methoxynicotinamide (263 mg) as a white powder.

1H-NMR (DMSO-d6): δ1.06-1.19 (2H, m), 1.35-1.52 (2H, m), 1.74-1.92 (5H, m), 2.48-2.58 (1H, m), 3.20-3.46 (1H, br), 3.79-3.90 (1H, m), 3.93 (3H, s), 6.62 (1H, d, J=7.8 Hz), 7.29-7.39 (2H, m), 7.93 (1H, s)

MS (ESI, m/z): 299 (M+H)

### Preparation 20

The following compound was obtained in a similar manner to that of Preparation 18:

### tert-butyl {trans-4-[(3-bromo-5-carbamoyl-6-methoxypyridin-2-yl)amino]cyclohexyl}carbamate

1H-NMR (DMSO-d6): δ1.18-1.56 (13H, m), 1.77-1.87 (2H, m), 1.87-1.97 (2H, m), 3.14-3.28 (1H, m), 3.73-3.88 (1H, m), 3.93 (3H, s), 6.39 (1H, d, J=7.8 Hz), 6.75 (1H, d, J=7.8 Hz), 7.29-7.39 (2H, m), 8.08 (1H, s)

MS (ESI, m/z): 444 (M+H)

### Example 5

The following compound was obtained in a similar manner to that of Example 4:

### 6-[(trans-4-aminocyclohexyl)amino]-5-bromo-2-methoxynicotinamide

1H-NMR (DMSO-d6): δ 1.07-1.21 (2H, m), 1.39-1.52 (2H, m), 1.73-1.94 (5H, m), 2.49-2.60 (1H, m), 3.04-3.60 (1H, br), 3.78-3.89 (1H, m), 3.93 (3H, s), 6.33 (1H, d, J=7.8 Hz), 7.28-7.41 (2H, m), 8.07 (1H, s)

MS (ESI, m/z): 344 (M+H)

### Preparation 21

The following compound was obtained in a similar manner to that of Preparation 2:

### tert-butyl {trans-4-[(5-cyano-6-ethoxypyridin-2-yl)amino]cyclohexyl}carbamate

1H-NMR (DMSO-d6): δ 1.20-1.25 (4H, m), 1.28 (3H, t, J=7.0 Hz), 1.38 (9H, s), 1.80 (2H, br s), 1.95 (2H, br s), 3.22 (1H, br s), 3.65 (1H, br s), 4.34 (2H, q, J=7.0 Hz), 6.07 (1H, d, J=8.6 Hz), 6.75 (1H, d, J=7.8 Hz), 7.50-7.55 (2H, m). MS (ESI, m/z): 383 (M+Na)

### Preparation 22

The following compound was obtained in a similar manner to that of Preparation 7:

### tert-butyl {trans-4-[(5-carbamoyl-6-ethoxypyridin-2-yl)amino]cyclohexyl}carbamate

1H-NMR (DMSO-d6): δ 1.20-1.35 (4H, m), 1.35 (3H, t, J=7.1 Hz), 1.38 (9H, s), 1.81 (2H, m), 1.99 (2H, m), 3.22 (1H, m), 3.60 (1H, m), 4.39 (2H, q, J=7.1 Hz), 6.07 (1H, d, J=8.5 Hz), 6.74 (1H, d, J=7.9 Hz), 7.05 (1H, br s), 7.12-7.25 (2H, m), 7.86 (1H, d, J=8.5 Hz)

MS (ESI, m/z): 401 (M+Na)

### Example 6

The following compound was obtained in a similar manner to that of Example 1:

### 6-[(trans-4-aminocyclohexyl)amino]-2-ethoxynicotinamide dihydrochloride

1H-NMR (DMSO-d6): δ 1.37 (3H, t, J=6.7 Hz), 1.20-1.50 (4H, m), 1.95-2.00 (4H, m), 3.00 (1H, m), 3.43 (1H, m), 4.41 (2H, q, J=6.7 Hz), 6.10 (1H, d, J=8.5 Hz), 7.10 (3H, br m), 7.88 (1H, d, J=8.5 Hz), 8.00 (3H, m).

MS (ESI, m/z): 279 (M+H)

### Preparation 23

The following compound was obtained in a similar manner to that of Preparation 3:

### tert-butyl {trans-4-[(5-cyano-6-ethoxy-3-fluoropyridin-2-yl)amino]cyclohexyl}carbamate

1H-NMR (DMSO-d6): δ1.20-1.45 (4H, m), 1.32 (3H, t, J=7.0 Hz), 1.38 (9H, s), 1.80-1.95 (4H, m), 3.20 (1H, m), 0.378 (1H, m), 4.32 (2H, q, J=7.0 Hz), 6.74 (1H, d, J=7.8 Hz), 7.58 (1H, d, J=7.4 Hz), 7.71 (1H, d, J=10.8 Hz).

MS (ESI, m/z): 401 (M+Na)

### Preparation 24

The following compound was obtained in a similar manner to that of Preparation 7:

### tert-butyl {trans-4-[(5-carbamoyl-6-ethoxy-3-fluoropyridin-2-yl)amino]cyclohexyl}carbamate

1H-NMR (DMSO-d6): δ 1.95-1.40 (4H, m), 1.36 (3H, t, J=7.1 Hz), 1.38 (9H, s), 1.80-1.85 (2H, m), 1.91-1.95 (2H, m), 3.20 (1H, m), 3.75 (1H, m), 4.38 (2H, q, J=7.1 Hz), 6.74 (1H, d, J=8.0 Hz), 7.10 (1H, d, J=7.7 Hz), 7.33 (2H, m), 7.68 (1H, d, J=11.7 Hz).

ESI-MS (m/z): 419 (M+Na)

### Example 7

The following compound was obtained in a similar manner to that of Example 1:

### 6-[(trans-4-aminocyclohexyl)amino]-2-ethoxy-5-fluoronicotinamide dihydrochloride

1H-NMR (DMSO-d6): δ 1.37 (3H, t, J=7.0 Hz), 1.32-1.42 (4H, m), 2.00-2.10 (4H, m), 2.97 (1H, br s), 3.77 (1H, br s), 3.40 (2H, q, J=7.0 Hz), 7.11-7.46 (3H, m), 7.71 (1H, d, J=11.7 Hz), 8.22 (3H, m).

ESI-MS (m/z): 297 (M+H)

### Preparation 25

The following compound was obtained in a similar manner to that of Preparation 8:

### tert-butyl {trans-4-[(5-cyano-6-ethoxypyridin-2-yl)amino]cyclohexyl}carbamate

1H-NMR (DMSO-d6): δ 1.20-1.25 (4H, m), 1.28 (3H, t, J=7.0 Hz), 1.38 (9H, s), 1.80 (2H, br s), 1.95 (2H, br s), 3.22 (1H, br s), 3.65 (1H, br s), 4.34 (2H, q, J=7.0 Hz), 6.07 (1H, d, J=8.6 Hz), 6.75 (1H, d, J=7.8 Hz), 7.150-7.55 (2H, m).

ESI-MS (m/z): 383 (M+Na)

### Preparation 26

The following compound was obtained in a similar manner to that of Preparation 18:

### tert-butyl {trans-4-[(3-chloro-5-cyano-6-ethoxypyridin-2-yl)amino]cyclohexyl}carbamate

1H-NMR (DMSO-d6): δ 1.16-1.43 (14H, m), 1.43-1.57 (2H, m), 1.76-1.91 (4H, m), 3.12-3.27 (1H, m), 3.76-3.88 (1H, m), 4.36 (2H, q, J=7.0 Hz), 6.74 (1H, d, J=7.8 Hz), 7.11 (1H, d, J=7.8 Hz), 7.91-7.93 (1H, m)

ESI-MS (m/z): 417 (M+Na)

### Preparation 27

The following compound was obtained in a similar manner to that of Preparation 7:

### tert-butyl {trans-4-[(5-carbamoyl-3-chloro-6-ethoxypyridin-2-yl)amino]cyclohexyl}carbamate

1H-NMR (DMSO-d6): δ1.17-1.56 (16H, m), 1.76-1.96 (4H, m), 3.14-3.28 (1H, m), 3.74-3.86 (1H, m), 4.42 (2H, q, J=7.0 Hz), 6.67 (1H, d, J=7.8 Hz), 6.74 (1H, d, J=7.8 Hz), 7.24-7.32 (1H, m), 7.34-7.41 (1H, m), 7.94 (1H, s)

MS (ESI, m/z): 435 (M+Na)

### Example 8

The following compound was obtained in a similar manner to that of Example 4:

### 6-[(trans-4-aminocyclohexyl)amino]-5-chloro-2-ethoxynicotinamide

1H-NMR (DMSO-d6): δ1.05-1.18 (2H, m), 1.33-1.51 (6H, m), 1.74-1.91 (4H, m), 2.47-2.58 (1H, m), 3.10-3.55 (1H, br), 3.75-3.87 (1H, m), 4.42 (2H, q, J=7.0 Hz), 6.61 (1H, d, J=7.8 Hz), 7.29 (1H, s), 7.36 (1H, s), 7.93 (1H, s)

MS (ESI, m/z): 313 (M+H)

### Preparation 28

The following compound was obtained in a similar manner to that of Preparation 18:

### tert-butyl {trans-4-[(3-bromo-5-cyano-6-ethoxypyridin-2-yl)amino]cyclohexyl}carbamate

1H-NMR (DMSO-d6): δ 1.17-1.42 (14H, m), 1.45-158 (2H, m), 1.76-1.91 (4H, m), 3.12-3.27 (1H, m), 3.74-3.87 (1H, m), 4.36 (2H, q, J=7.0 Hz), 6.74 (1H, d, J=7.8 Hz), 6.81 (1H, d, J=7.8 Hz), 8.02-8.05 (1H, m)

MS (ESI, m/z) 462 (M+Na)

### Preparation 29

The following compound was obtained in a similar manner to that of Preparation 7:

### tert-butyl {trans-4-[(3-bromo-5-carbamoyl-6-ethoxypyridin-2-yl)amino]cyclohexyl}carbamate

1H-NMR (DMSO-d6): δ 1.18-1.56 (16H, m), 1.77-1.95 (4H, m), 3.12-3.29 (1H, m), 3.73-3.85 (1H, m), 4.42 (2H, q, J=7.0 Hz), 6.38 (1H, d, J=7.8 Hz), 6.73 (1H, d, J=7.8 Hz), 7.23-7.32 (1H, m), 7.33-7.42 (1H, m), 8.08 (1H, s)

MS (ESI, m/z) 458 (M+H)

### Example 9

The following compound was obtained in a similar manner to that of Example 4:

### 6-[(trans-4-aminocyclohexyl)amino]-5-bromo-2-ethoxynicotinamide

1H-NMR (DMSO-d6): δ1.05-1.18 (2H, m), 1.34-1.52 (6H, m), 1.74-1.91 (4H, m), 2.49-2.58 (1H, m), 3.23-3.44 (1H, br), 3.74-3.86 (1H, m), 4.42 (2H, q, J=7.0 Hz), 6.31 (1H, d, J=7.8 Hz), 7.28 (1H, s), 7.37 (1H, s), 8.08 (1H, s)

MS (ESI, m/z): 358 (M+H)

### Preparation 30

The following compound was obtained in a similar manner to that of Preparation 18:

### tert-butyl {trans-4-[(3-iodo-5-cyano-6-ethoxypyridin-2-yl)amino]cyclohexyl}carbamate

1H-NMR (DMSO-d6): δ1.14-1.43 (14H, m), 1.44-158 (2H, m), 1.76-1.92 (4H, m), 3.14-3.28 (1H, m), 3.71-3.83 (1H, m), 4.36 (2H, q, J=7.0 Hz), 6.30 (1H, d, J=7.7 Hz), 6.73 (1H, d, J=7.7 Hz), 8.11-8.13 (1H, m)

MS (ESI, m/z): 509 (M+H)

### Preparation 31

The following compound was obtained in a similar manner to that of Preparation 7:

### tert-butyl {trans-4-[(3-iodo-5-carbamoyl-6-ethoxypyridin-2-yl)amino]cyclohexyl}carbamate

1H-NMR (DMSO-d6): δ1.18-1.55 (16H, m), 1.77-1.86 (2H, m), 1.87-1.96 (2H, m), 3.15-3.29 (1H, m), 3.69-3.81 (1H, m), 4.41 (2H, q, J=7.0 Hz), 5.91 (1H, d, J=7.8 Hz), 6.73 (1H, d, J=7.8 Hz), 7.21-7.29 (1H, m), 7.30-7.38 (1H, m), 8.27 (1H, s)

MS (ESI, m/z): 527 (M+Na)

### Example 10

The following compound was obtained in a similar manner to that of Example 4:

### 6-[(trans-4-aminocyclohexyl)amino]-5-iodo-2-ethoxynicotinamide

1H-NMR (DMSO-d6): δ1.06-1.18 (2H, m), 1.34-1.50 (6H, m), 1.74-1.83 (2H, m), 1.84-1.93 (2H, m), 2.49-2.59 (1H, m), 3.16-3.46 (1H, br), 3.70-3.82 (1H, m), 4.42 (2H, q, J=7.0 Hz), 5.83 (1H, d, J=7.7 Hz), 7.24 (1H, s), 7.34 (1H, s), 8.27 (1H, s)

MS (ESI, m/z): 305 (M+H)

### Preparation 32

To a solution of tert-butyl {trans-4-[(3-iodo-5-cyano-6-ethoxypyridin-2-yl)amino]cyclohexyl}carbamate (440 mg) and trimethylboroxine (138 mg) in 1,4-dioxane (15 mL) were added potassium carbonate (375 mg) and tetrakis(triphenylphospine) palladium (52 mg) and the mixture was stirred at 140° C. for 14 hours. The mixture was evaporated in vacuo and the residue was purified by column chromatography on silica gel eluting with hexane:ethyl acetate (1:1) to afford tert-butyl {trans-4-[(5-cyano-6-ethoxy-3-methylpyridin-2-yl)amino]cyclohexyl}carbamate (263 mg) as a white powder.

1H-NMR (DMSO-d6): δ1.18-1.49 (16H, m), 1.77-1.87 (2H, m), 1.87-2.00 (5H, m), 3.15-3.28 (1H, m), 3.75-3.88 (1H, m), 4.33 (2H, q, J=7.0 Hz), 6.55 (1H, d, J=7.7 Hz), 6.73 (1H, d, J=7.7 Hz), 7.40-7.44 (1H, m)

MS (ESI, m/z): 397 (M+Na)

### Preparation 33

The following compound was obtained in a similar manner to that of Preparation 7:

### tert-butyl {trans-4-[(3-methyl-5-carbamoyl-6-ethoxypyridin-2-yl)amino]cyclohexyl}carbamate

1H-NMR (DMSO-d6): δ1.18-1.46 (16H, m), 1.77-1.87 (2H, m), 1.90-2.02 (5H, m), 3.14-3.28 (1H, m), 3.73-3.86 (1H, m), 4.39 (2H, q, J=7.0 Hz), 6.12 (1H, d, J=7.7 Hz), 7.73 (1H, d, J=7.7 Hz), 7.07-7.17 (1H, m), 7.21-7.32 (1H, m), 7.70-7.74 (1H, m)

MS (ESI, m/z): 415 (M+Na)

### Example 11

The following compound was obtained in a similar manner to that of Example 4:

### 6-[(trans-4-aminocyclohexyl)amino]-5-methyl-2-ethoxynicotinamide

1H-NMR (DMSO-d6): δ1.05-1.18 (2H, m), 1.30-1.50 (6H, m), 1.74-1.83 (2H, m), 1.86-1.93 (2H, m), 1.98 (3H, s), 2.48-2.58 (1H, m), 3.22-3.43 (1H, br), 3.75-3.87 (1H, m), 4.40 (2H, q, J=7.0 Hz), 6.08 (1H, d, J=7.6 Hz), 7.11 (1H, s), 7.25 (1H, s), 7.71 (1H, s)

MS (ESI, m/z): 293 (M+H)

### Preparation 34

The following compound was obtained in a similar manner to that of Preparation 2:

### tert-butyl {trans-4-[(5-cyano-3-fluoro-6-isopropoxypyridin-2-yl)amino]cyclohexyl}carbamate

1H-NMR (DMSO-d6): δ1.22-1.32 (4H, m), 1.36 (6H, d, J=6.2 Hz), 1.45 (9H, s), 2.10-2.15 (4H, m), 3.48 (1H, m), 3.81 (1H, m), 4.41 (1H, m), 4.86 (1H, d, J=5.2 Hz), 5.16 (1H, sept), 7.21 (1H, d, J=10.0 Hz).

MS (ESI, m/z): 415 (M+Na)

### Preparation 35

The following compound was obtained in a similar manner to that of Preparation 7:

### tert-butyl {trans-4-[(5-carbamoyl-3-fluoro-6-isopropoxypyridin-2-yl)amino]cyclohexyl}carbamate

1H-NMR (DMSO-d6): δ 1.23-1.29 (4H, m), 1.36 (6H, d, J=6.2 Hz), 1.45 (9H, s), 1.80-1.83 (2H, m), 1.91-1.93 (2H, m), 3.22 (1H, m), 3.75 (1H, m), 5.22 (1H, sept, J=6.2 Hz), 6.74 (1H, d, J=8.0 Hz), 7.12 (1H, d, J=7.4 Hz), 7.30 (1H, s), 7.36 (1H, s), 7.68 (1H, d, J=11.7 Hz).

MS (ESI, m/z): 433 (M+Na)

### Example 12

The following compound was obtained in a similar manner to that of Example 1:

### 6-[(trans-4-aminocyclohexyl)amino]-5-fluoro-2-isopropoxynicotinamide dihydrochloride

1H-NMR (DMSO-d6): δ1.28 (6H, d, J=6.2 Hz), 1.25-1.38 (4H, m), 1.85-2.00 (4H, m), 3.00 (1H, m), 3.75 (1H, m), 5.24 (1H, sept, J=6.2 Hz), 7.20-7.39 (3H, m), 7.71 (1H, d, J=10.1 Hz), 8.33 (3H, s)

MS (ESI, m/z): 311 (M+Na)

### Preparation 36

To a solution tert-butyl {trans-4-[(6-chloro-5-cyano-3-fluoropyridin-2-yl)amino]cyclohexyl}carbamate (310 mg) and 2-fluoroethanol (135 mg) in toluene (9.3 mL) were added sodium hydroxide (67 mg) and Cetyltrimethylammonium bromide (31 mg) and the mixture was stirred at 100° C. for 4 hours under nitrogen. The mixture was concentrated in vacuo and water (10 mL) was added. The mixture was adjusted to pH 5 by addition of 1N hydrochloric acid and the resulting precipitates were collected by filtration, washed with water, dried in vacuo to afford tert-butyl (trans-4-{[5-cyano-3-fluoro-6-(2-fluoroethoxy)pyridin-2-yl]amino}cyclohexyl)carbamate (315 mg) as white crystals.

1H-NMR (DMSO-d6): δ 1.23-1.30 (4H, m), 1.38 (9H, s), 1.82-1.90 (4H, m), 3.20 (1H, m), 3.77 (1H, m), 4.53 (2H, dt, J=4.1, 29.8 Hz), 4.75 (2H, dq, J=3.8, 47.7 Hz), 6.76 (1H, d, J=7.8 Hz), 7.63 (1H, d, J=7.1 Hz), 7.75 (1H, d, J=10.8 Hz).

MS (ESI, m/z): 419 (M+Na)

### Preparation 37

The following compound was obtained in a similar manner to that of Preparation 7:

### tert-butyl (trans-4-{[5-carbamoyl-3-fluoro-6-(2-fluoroethoxy)pyridin-2-yl]amino}cyclohexyl)carbamate

1H-NMR (DMSO-d6): δ1.20-1.38 (4H, m), 1.38 (9H, s), 1.80-1.93 (4H, m), 3.20 (1H, m), 3.75 (1H, m), 4.75 (2H, dt, J=4.0, 29.4 Hz), 4.83 (2H, dt, J=4.0, 47.6 Hz), 6.76 (1H, d, J=7.8 Hz), 7.17 (1H, d, J=7.1 Hz), 7.28 (1H, s), 7.41 (1H, s), 7.71 (1H, d, J=11.6 Hz).

MS (ESI, m/z): 437 (M+Na)

### Example 13

To a solution of tert-butyl (trans-4-{[(5-carbamoyl-3-fluoro-6-(2-fluoroethoxy)pyridin-2-yl]amino}cyclohexyl)carbamate (90 mg) in dichloromethane (0.9 ml) was added trifluoroacetic acid (198 mg) and the mixture was stirred at ambient temperature for 5 hours. The resulting precipitates were collected by filtration and washed with ethyl acetate, and dried in vacuo to afford 6-[(trans-4-aminocyclohexyl)amino]-5-fluoro-2-(2-fluoroethoxy)nicotinamide bis(trifluoroacetate) (90 mg) as white crystals.

1H-NMR (DMSO-d6): δ 1.30-1.47 (4H, m), 1.97-2.05 (4H, m), 3.00 (1H, m), 3.76 (1H, m), 4.58 (2H, dt, J=4.0, 29.2 Hz), 4.81 (2H, dt, J=4.0, 47.6 Hz), 7.25 (1H, d, J=7.4 Hz), 7.29 (1H, s), 7.44 (1H, s), 7.73 (1H, d, J=11.7 Hz), 7.80 (3H, m).

MS (ESI, m/z): 337 (M+Na), 315 (M+H)

### Preparation 38

The following compound was obtained in a similar manner to that of Preparation 36:

### tert-butyl (trans-4-{[5-cyano-6-(2,2-difluoroethoxy)-3-fluoropyridin-2-yl]amino}cyclohexyl)carbamate

1H-NMR (DMSO-d6) δ 1.25-1.41 (4H, m), 1.39 (9H, s), 1.80-1.92 (4H, m), 3.20 (1H, m), 3.77 (1H, m), 4.59 (2H, dt, J=3.6, 14.7 Hz), 6.39 (1H, tt, J=3.6, 54.6 Hz), 6.77 (1H, d, J=7.8 Hz), 7.74 (1H, d, J=7.3 Hz), 7.78 (1H, d, J=10.7 Hz).

MS (ESI, m/z): 437 (M+Na)

### Preparation 39

The following compound was obtained in a similar manner to that of Example 7:

### tert-butyl(trans-4-{[5-carbamoyl-6-(2,2-difluoroethoxy)-3-fluoropyridin-2-yl]amino}cyclohexyl)carbamate

1H-NMR (DMSO-d6) δ 1.24-1.38 (4H, m), 1.38 (9H, s), 1.80-1.93 (4H, m), 3.20 (1H, m), 3.75 (1H, m), 4.63 (2H, dt, J=3.4, 14.9 Hz), 6.48 (1H, tt, J=3.4, 54.9 Hz), 6.77 (1H, d, J=7.8 Hz), 7.20 (1H, s), 7.26 (1H, d, J=7.3 Hz), 7.45 (1H, s), 7.72 (1H, d, J=11.52 Hz).

MS (ESI, m/z): 455 (M+Na)

### Example 14

The following compound was obtained in a similar manner to that of Example 1:

### 6-[(trans-4-aminocyclohexyl)amino]-2-(2,2-difluoroethoxy)-5-fluoronicotinamide dihydrochloride

1H-NMR (DMSO-d6): δ1.25-1.35 (4H, m), 1.91-2.05 (4H, m), 2.95 (1H, m), 3.80 (1H, m), 4.65 (2H, dt, J=3.6, 14.8 Hz), 6.50 (1H, tt, J=3.6, 55.0 Hz), 7.22 (1H, br s), 7.34 (1H, br s), 7.49 (1H, br s), 7.73 (1H, d, J=11.5 Hz), 8.16 (4H, m).

MS (ESI, m/z): 333 (M+H)

### Preparation 40

The following compound was obtained in a similar manner to that of Preparation 36:

### tert-butyl (trans-4-{[5-cyano-3-fluoro-6-(2,2,2-trifluoroethoxy)pyridin-2-yl]amino}cyclohexyl)carbamate

1H-NMR (DMSO-d6): δ1.21-1.31 (4H, m), 1.41 (9H, s), 1.80-1.90 (4H, m), 3.02 (1H, br s), 3.80 (1H, br s), 5.02 (2H, q, J=9.0 Hz), 6.77 (1H, d, J=7.7 Hz), 7.80 (1H, m), 7.82 (1H, d, J=11.7 Hz).

MS (ESI, m/z): 455 (M+Na)

### Preparation 41

The following compound was obtained in a similar manner to that of Preparation 7:

### tert-butyl (trans-4-{[5-carbamoyl-3-fluoro-6-(2,2,2-trifluoroethoxy)pyridin-2-yl]amino}cyclohexyl)carbamate

1H-NMR (DMSO-d6): δ 1.25-1.42 (4H, m), 1.38 (9H, s), 1.79-1.92 (4H, m), 3.22 (1H, br s), 3.77 (1H, m), 5.07 (2H, q, J=9.0 Hz), 6.77 (1H, d, J=7.9°Hz), 7.08 (1H, s), 7.32 (1H, d, J=7.4 Hz), 7.50 (1H, s), 7.72 (1H, d, J=11.4 Hz).

MS (ESI, m/z): 473 (M+Na)

### Example 15

The following compound was obtained in a similar manner to that of Example 1:

### 6-[(trans-4-aminocyclohexyl)amino]-5-fluoro-2-(2,2,2-trifluoroethoxy)nicotinamide dihydrochloride

1H-NMR (DMSO-d6): δ 1.35-1.50 (4H, m), 1.95-2.05 (4H, m), 3.00 (1H, m), 3.80 (1H, m), 5.08 (1H, q, J=9.0 Hz), 7.09 (1H, s), 7.39 (1H, d, J=7.5 Hz), 7.52 (1H, s), 7.73 (1H, d, J=11.4 Hz), 8.10 (4H, m).

MS (ESI, m/z): 351 (M+H) Preparation 42

The following compound was obtained in a similar manner to that of Preparation 36:

### tert-butyl {trans-4-[(5-cyano-3-fluoro-6-isobutoxypyridin-2-yl)amino]cyclohexyl}carbamate

1H-NMR (DMSO-d6): δ 0.95 (6H, d, J=6.7 Hz), 1.18-1.46 (4H, m), 1.37 (9H, s), 1.76-1.93 (4H, m), 1.97-2.08 (1H, m), 3.12-3.27 (1H, m), 3.68-3.82 (1H, m), 4.07 (2H, d, J=6.5 Hz), 6.75 (1H, d, J=7.7 Hz), 7.59 (1H, d, J=7.6 Hz), 7.70 (1H, d, J=10.8 Hz).

MS (ESI, m/z): 429 (M+Na)

### Preparation 43

The following compound was obtained in a similar manner to that of Preparation 7:

### tert-butyl {trans-4-[(5-carbamoyl-3-fluoro-6-isobutoxypyridin-2-yl)amino]cyclohexyl}carbamate

1H-NMR (DMSO-d6): δ 0.96 (6H, d, J=6.7 Hz), 1.19-1.45 (4H, m), 1.38 (9H, s), 1.78-1.96 (4H, m), 2.04-2.14 (1H, m), 3.15-3.28 (1H, m), 3.67-3.79 (1H, m), 4.16 (2H, d, J=6.6 Hz), 6.75 (1H, d, J=8.0 Hz), 7.13 (1H, d, J=7.2 Hz), 7.29 (1H, bs), 7.38 (1H, bs), 7.69 (1H, d, J=11.6 Hz).

MS (ESI, m/z): 447 (M+Na)

### Example 16

The following compound was obtained in a similar manner to that of Example 13:

### 6-[(trans-4-aminocyclohexyl)amino]-5-fluoro-2-isobutoxynicotinamide bis(trifluoroacetate)

1H-NMR (DMSO-d6): δ0.97 (6H, d, J: 6.7 Hz), 1.34-1.48 (4H, m), 1.92-2.15 (5H, m), 2.95-3.07 (1H, m), 3.70-3.81 (1H, m), 4.16 (2H, d, J=6.5 Hz), 7.22 (1H, d, J=7.1 Hz), 7.30 (1H, bs), 7.43 (1H, bs), 7.71 (1H, d, J=11.7 Hz), 7.82 (2H, bs).

MS (ESI, m/z): 325 (M+H),

### Preparation 44

The following compound was obtained in a similar manner to that of Preparation 36:

### tert-butyl [trans-4-({6-[2-(benzyloxy)ethoxy]-5-cyano-3-fluoropyridin-2-yl}amino)cyclohexyl]carbamate

1H-NMR (DMSO-d6): δ1.17-1.46 (4H, m), 1.38 (9H, s), 1.76-1.90 (4H, m), 3.12-3.26 (1H, m), 3.68-3.81 (1H, m), 3.74-3.77 (2H, m), 4.43-4.46 (2H, m), 4.56 (2H, s), 6.74 (1H, d, J=7.8 Hz), 7.24-7.37 (5H, m), 7.59 (1H, d, J=7.5 Hz), 7.73 (1H, d, J=10.8 Hz).

negative MS (ESI, m/z) 483 (M−H)

### Preparation 45

The following compound was obtained in a similar manner to that of Preparation 7:

### tert-butyl [trans-4-({6-[2-(benzyloxy)ethoxy]-5-carbamoyl-3-fluoropyridin-2-yl}amino)cyclohexyl]carbamate

1H-NMR (DMSO-d6): δ 1.18-1.44 (4H, m), 1.38 (9H, s), 1.77-1.94 (4H, m), 3.12-3.27 (1H, m), 3.68-3.78 (1H, m), 3.74-3.80 (2H, m), 4.47-4.49 (2H, m), 4.55 (2H, s), 6.74 (1H, d, J=7.9 Hz), 7.13 (1H, d, J=7.6 Hz), 7.26-7.38 (5H, m), 7.40 (1H, bs), 7.42 (1H, bs), 7.70 (1H, d, J=11.7 Hz).

MS (ESI, m/z): 525 (M+Na)

### Preparation 46

To a solution of tert-butyl [trans-4-({6-[2-(benzyloxy)ethoxy]-5-carbamoyl-3-fluoropyridin-2-yl}amino)cyclohexyl]carbamate (228 mg) in ethyl acetate (7 mL) and methanol (7 mL) was added 10% Palladium on charcoal (50% wet) (67 mg) and the mixture was hydrogenated at atmospheric pressure for 3 hours. The catalyst was filtered off on celite pad and the filtarte was evaporated in vacuo. The residue was triturated with diisopropylether to afford tert-butyl (trans-4-{[5-carbamoyl-3-fluoro-6-(2-hydroxyethoxy)pyridin-2-yl]amino}cyclohexyl)carbamate (167 mg) as a powder.

1H-NMR (DMSO-d6): δ 1.19-1.45 (4H, m), 1.38 (9H, s), 1.76-1.96 (4H, m), 3.12-3.27 (1H, m), 3.68-3.80 (3H, m), 4.32 (2H, t, J=4.8 Hz), 4.97 (1H, t, J=5.4 Hz), 6.73 (1H, d, J=7.8 Hz), 7.09 (1H, d, J=7.6 Hz), 7.34 (1H, bs), 7.45 (1H, bs), 7.69 (1H, d, J=11.6 Hz).

MS (ESI, m/z): 435 (M+Na)

### Example 17

The following compound was obtained in a similar manner to that of Example 4:

### 6-[(trans-4-aminocyclohexyl)amino]-5-fluoro-2-(2-hydroxyethoxy)nicotinamide

1H-NMR (DMSO-d6): δ1.06-1.18 (2H, m), 1.30-1.42 (2H, m), 1.74-1.91 (4H, m), 2.46-2.56 (1H, m), 3.70-3.81 (3H, m), 4.33 (2H, t, J=4.8 Hz), 4.97 (1H, bs), 7.07 (1H, d, J=7.4 Hz), 7.34 (1H, bs), 7.44 (1H, bs), 7.68 (1H, d, J=11.6 Hz).

MS (ESI, m/z): 330 (M+H)

### Preparation 47

The following compound was obtained in a similar manner to that of Preparation 8:

### benzyl {trans-4-[(5-cyano-6-methoxypyridin-2-yl)amino]cyclohexyl}carbamate

1H-NMR (DMSO-d6): δ 1.18-1.36 (4H, m), 1.77-2.07 (4H, m), 3.24-3.38 (1H, m), 3.60-3.79 (1H, m), 3.87 (3H, s), 5.00 (2H, s), 6.10 (1H, d, J=7.7 Hz), 7.23 (1H, d, J=7.7 Hz), 7.28-7.40 (5H, m), 7.48-7.67 (2H, br)

MS (ESI, m/z): 381 (M+H)

### Preparation 48

The following compound was obtained in a similar manner to that of Preparation 18:

### benzyl {trans-4-[(5-cyano-3-iodo-6-methoxypyridin-2-yl)amino]cyclohexyl}carbamate

1H-NMR (DMSO-d6): δ1.21-1.36 (2H, m), 1.46-1.61 (2H, m), 1.81-1.95 (4H, m), 3.23-3.36 (1H, m), 3.76-3.87 (1H, m), 3.89 (3H, s), 5.01 (2H, s), 6.32 (1H, d, J=7.7 Hz), 7.21 (1H, d, J=7.7 Hz), 7.28-7.41 (5H, m), 8.13 (1H, s)

MS (ESI, m/z): 507 (M+H)

### Preparation 49

The following compound was obtained in a similar manner to that of Preparation 32:

### benzyl {trans-4-[(5-cyano-6-methoxy-3-methylpyridin-2-yl)amino]cyclohexyl}carbamate

1H-NMR (DMSO-d6): δ1.22-1.36 (2H, m), 1.37-1.51 (2H, m), 1.82-2.00 (7H, m), 3.25-3.36 (1H, m), 3.81-3.93 (4H, m), 5.01 (2H, s), 6.58 (1H, d, J=7.7 Hz), 7.22 (1H, d, J=7.7 Hz), 7.28-7.41 (5H, m), 7.42-7.46 (1H, m)

MS (ESI, m/z): 395 (M+H)

### Preparation 50

The following compound was obtained in a similar manner to that of Preparation 7:

### benzyl {trans-4-[(5-carbamoyl-6-methoxy-3-methylpyridin-2-yl)amino]cyclohexyl}carbamate

1H-NMR (DMSO-d6): δ1.22-1.50 (4H, m), 1.82-2.03 (7H, m), 3.24-3.36 (1H, m), 3.77-3.94 (4H, m), 5.01 (2H, s), 6.13 (1H, d, J=7.7 Hz), 7.03-7.14 (1H, br), 7.21 (1H, d, J=7.7 Hz), 7.24-7.45 (6H, m), 7.70-7.74 (1H, m)

MS (ESI, m/z): 413 (M+H)

### Example 18

The following compound was obtained in a similar manner to that of Example 3:

### 6-[(trans-4-aminocyclohexyl)amino]-2-methoxy-5-methylnicotinamide

1H-NMR (DMSO-d6): δ1.06-1.20 (2H, m), 1.29-1.60 (4H, m), 1.74-1.84 (2H, m), 1.87-2.02 (5H, m), 2.46-2.59 (1H, m), 3.79-3.94 (4H, m), 6.08 (1H, d, J=7.7 Hz), 7.03-7.15 (1H, br), 7.22-7.32 (1H, br), 7.71 (1H, s)

MS (ESI, m/z): 279 (M+H)

### Preparation 51

The following compound was obtained in a similar manner to that of Preparation 1:

### methyl 6-({trans-4-[(tert-butoxycarbonyl)amino]cyclohexyl}amino)-2-chloro-5-fluoronicotinate

1H-NMR (DMSO-d6): δ 1.17-1.31 (4H, m), 1.38 (9H, s), 1.82 (4H, t, J=12.6 Hz), 3.20 (1H, brs), 3.77 (3H, s), 3.81 (1H, brs), 6.77 (1H, d, J=7.74 Hz), 7.69 (1H, d, J=7.14 Hz), 7.77 (1H, d, J=11.3 Hz)

### Preparation 52

The following compound was obtained in a similar manner to that of Preparation 3:

### 6-({trans-4-[(tert-butoxycarbonyl)amino]cyclohexyl}amino)-2-chloro-5-fluoronicotinic acid

1H-NMR (DMSO-d6): δ1.19-1.31 (4H, m), 1.38 (9H, s), 1.83 (4H, t, J=11.3 Hz), 3.20 (1H, brs), 3.77 (1H, brs), 6.77 (1H, d, J=7.86 Hz), 7.57 (1H, d, J=8.25 Hz), 7.74 (1H, d, J=11.2 Hz)

### Preparation 53

The following compound was obtained in a similar manner to that of Preparation 4:

### tert-butyl {trans-4-[(3-fluoro-5-carbamoyl-6-chloropyridin-2-yl)amino]cyclohexyl}carbamate

1H-NMR (DMSO-d6): δ 1.18-1.35 (4H, m), 1.38 (9H, s), 1.83 (4H, t, J=13.2 Hz), 3.12-3.28 (1H, m), 3.68-3.78 (1H, m), 6.76 (1H, d, J=7.92 Hz), 7.17 (1H, d, J=8.64 Hz), 7.47 (1H, s), 7.52 (1H, d, J=11 Hz), 7.65 (1H, s)

### Example 19

The following compound was obtained in a similar manner to that of Example 4:

### 6-[(trans-4-aminocyclohexyl)amino]-5-fluoro-2-chloronicotinamide

1H-NMR (DMSO-d6): δ 1.04-1.18 (2H, m), 1.28-1.42 (2H, m), 1.80 (4H, t, J=17.5 Hz), 3.20 (1H, brs), 3.73 (1H, brs), 7.13 (1H, brs), 7.45 (1H, brs), 7.52 (1H, d, J=11.2 Hz), 7.64 (1H, brs)

### Example 20

The following compound was obtained in a similar manner to that of Example 1:

### 6-[(trans-4-aminocyclohexyl)amino]-5-fluoro-2-chloronicotinamide dihydrochloride

1H-NMR (DMSO-d6): δ 1.35-1.51 (4H, m), 1.91-2.01 (4H, m), 2.96 (1H, m), 3.78 (1H, m), 7.27 (1H, br s), 7.49 (1H, br s), 7.55 (1H, d, J=10.8 Hz), 7.67 (1H, br s), 8.16 (3H, br s)

### Preparation 54

The following compound was obtained in a similar manner to that of Preparation 1:

### tert-butyl {trans-4-[(3-chloro-5-cyanopyridin-2-yl)amino]cyclohexyl}carbamate

1H-NMR (DMSO-d6): δ 1.13-1.31 (2H, m), 1.36 (9H, s), 1.38-1.56 (2H, m), 1.77 (2H, s), 1.80 (2H, s), 3.12-3.22 (1H, m), 3.85-3.97 (1H, m), 6.74 (1H, d, J=8.1 Hz), 7.11 (1H, d, J=8.1 Hz), 8.04, (1H, d, J=1.8 Hz), 8.40 (1H, d, J=2.1 Hz)

### Preparation 55

The following compound was obtained in a similar manner to that of Preparation 7:

### tert-butyl {trans-4-[(3-chloro-5-carbamoylpyridin-2-yl)amino]cyclohexyl}carbamate

1H-NMR (DMSO-d6): δ 1.17-1.50 (4H, m), 1.36 (9H, s), 1.80 (4H, t, J=12.2 Hz), 3.14-3.23 (1H, m), 3.84-3.93 (1H, m), 6.54 (1H, d, J=7.5 Hz), 6.74 (1H, d, J=7.2 Hz), 7.20 (1H, s), 7.77 (1H, s), 7.97 (1H, d, J=2.1 Hz), 8.48 (1H, d, J=2.1 Hz)

### Example 21

The following compound was obtained in a similar manner to that of Example 4:

### 6-[(trans-4-aminocyclohexyl)amino]-5-chloronicotinamide

1H-NMR (DMSO-d6): δ 1.05-1.18 (2H, m), 1.36-1.50 (2H, m), 1.79 (4H, t, J=13.6 Hz), 3.17 (1H, s), 3.86-3.99 (1H, m), 6.47 (1H, d, J=7.8 Hz), 7.23 (1H, s), 7.78 (1H, s), 7.99 (1H, d, J=1.8 Hz), 8.50 (1H, d, J=1.8 Hz)

### Preparation 56

The following compound was obtained in a similar manner to that of Preparation 1:

### tert-butyl {trans-4-[(5-cyanopyridin-2-yl)amino]cyclohexyl}carbamate

1H-NMR (DMSO-d6): δ 1.24 (4H, t, J=9.96 Hz), 1.38 (9H, s), 1.79 (2H, brs), 1.93 (2H, brs), 3.16-3.29 (1H, m), 3.62-3.78 (1H, m), 6.50 (1H, d, J=8.82 Hz), 6.76 (1H, d, J=8.16 Hz), 7.49 (1H, d, J=7.56 Hz), 7.64 (1H, dd, J=7.35, 2.25 Hz), 8.37 (1H, d, J=2.19 Hz)

### Preparation 57

The following compound was obtained in a similar manner to that of Preparation 7:

### tert-butyl {trans-4-[(5-carbamoylpyridin-2-yl)amino]cyclohexyl}carbamate

1H-NMR (DMSO-d6): δ 1.15-1.32 (4H, m), 1.38 (9H, s), 1.78 (2H, brs), 1.95 (2H, brs), 3.22 (1H, brs), 3.67 (1H, brs), 6.40 (1H, d, J=8.79 Hz), 6.77 (1H, d, J=8.19 Hz), 6.92 (1H, d, J=7.74 Hz), 7.03 (1H, brs), 7.63 (1H, brs), 7.77 (1H, dd, J=10.3, 2.37 Hz), 8.48 (1H, d, J=2.34 Hz)

### Example 22

The following compound was obtained in a similar manner to that of Example 4:

### 6-[(trans-4-aminocyclohexyl)amino]nicotinamide

1H-NMR (DMSO-d6): δ 1.03-1.27 (4H, brs), 1.74-1.92 (4H, m), 3.22 (1H, brs), 3.66 (1H, brs), 6.39 (1H, d, J=8.79 Hz), 6.88 (1H, d, J=7.74 Hz), 7.01 (1H, brs), 7.62 (1H, brs), 7.76 (1H, dd, J=8.79, 2.43 Hz), 8.49 (1H, d, J=2.31 Hz)

### Preparation 58

The following compound was obtained in a similar manner to that of Preparation 1:

### tert-butyl {trans-4-[(3,6-difluoro-5-carbamoylpyridin-2-yl)amino]cyclohexyl}carbamate

1H-NMR (DMSO-d6): δ (9H, s), 1.78-1.86 (4H, m), 3.20 (1H, m), 3.70 (1H, m), 6.76 (1H, d, J=8.0 Hz), 7.25 (1H, br s), 7.41-7.45 (2H, m), 7.69-7.72 (1H, m)

MS (ESI, m/z): 393 (M+Na)

### Example 23

The following compound was obtained in a similar manner to that of Example 1:

### 6-[(trans-4-aminocyclohexyl)amino]-2,5-difluoronicotinamide dihydrochloride

1H-NMR (DMSO-d6): δ1.36-1.52 (4H, m), 1.91-2.02 (4H, m), 2.90-3.00 (1H, m), 3.72 (1H, br s), 6.50-7.51 (4H, m), 7.70-7.75 (1H, m), 8.18 (3H, m)

MS (ESI, m/z): 271 (M+H)

### Preparation 59

The following compound was obtained in a similar manner to that of Preparation 1:

### tert-butyl {trans-4-[(5-cyano-6-chloropyridin-2-yl)amino]cyclohexyl}carbamate

1H-NMR (DMSO-d6): δ 1.20-1.30 (4H, m), 1.38 (9H, s), 1.79 (2H, br s), 1.89 (2H, br s), 3.23 (1H, br s), 3.65 (1H, br s), 6.47 (1H, d, J=8.16 Hz), 6.76 (1H, d, J=7.72 Hz), 7.70 (1H, d, J=8.16 Hz), 7.96 (1H, d, J=7.52 Hz)

MS (ESI, m/z): 373 (M+Na)

### Preparation 60

The following compound was obtained in a similar manner to that of Preparation 18:

### tert-butyl {trans-4-[(5-cyano-3,6-dichloropyridin-2-yl)amino]cyclohexyl}carbamate

1H-NMR (DMSO-d6): δ 1.18-1.31 (2H, m), 1.39 (9H, s), 1.46-1.58 (2H, m), 1.75-1.84 (4H, m), 3.14-3.26 (1H, m), 3.78-2.9 (1H, m), 6.75 (1H, d, J=7.9 Hz), 7.56 (1H, d, J=7.9 Hz), 8.17 (1H, s)

MS (ESI, m/z): 407 (M+Na)

### Preparation 61

The following compound was obtained in a similar manner to that of Preparation 7:

### tert-butyl {trans-4-[(5-carbamoyl-3,6-dichloropyridin-2-yl)amino]cyclohexyl}carbamate

1H-NMR (DMSO-d6): δ 1.19-1.32 (2H, m), 1.38 (9H, s), 1.4-1.54 (2H, m), 1.75-1.86 (4H, m), 3.15-3.26 (1H, m), 3.72-3.84 (1H, m), 6.75 (1H, d, J=7.9 Hz), 7.46 and 7.68 (total 2H, brs), 7.73 (1H, s)

MS (ESI, m/z): 425 (M+Na)

### Example 24

The following compound was obtained in a similar manner to that of Example 4:

### 6-[(trans-4-aminocyclohexyl)amino]-2,5-dichloronicotinamide

1H-NMR (DMSO-d6) δ 1.05-1.18 (2H, m), 1.38-1.5 (2H, m), 1.73-1.84 (4H, m), 3.74-3.86 (1H, m), 6.66 (1H, d, J=8.1 Hz), 7.45 and 7.67 (total 2H, brs), 7.73 (1H, s)

MS (ESI, m/z): 303 (M+H)

### Preparation 62

To a solution of tert-butyl {trans-4-[(6-chloro-5-cyano-3-fluoropyridin-2-yl)amino]cyclohexyl}carbamate (300 mg) in tetrahydrofuran (10 mL) was added 2M methylamine in tetrahydrofuran (20.3 mL) and the mixture was stirred at ambient temperature for 2 days. The mixture was evaporated in vacuo and the residue was poured into water and extracted with ethyl acetate. The organic layer was washed with brine, dried over magnesium sulfate, and evaporated in vacuo. The residue was purified by column chromatography on silica gel eluting with toluene:ethyl acetate (4:1) to afford tert-butyl {trans-4-[(3-fluoro-5-cyano-6-methylaminopyridin-2-yl)amino]cyclohexyl}carbamate (296 mg) as solids.

1H-NMR (DMSO-d6): δ 1.18-1.25 (2H, m), 1.33-1.42 (2H, m), 1.38 (9H, s), 1.78-1.81 (2H, m), 1.85-1.95 (2H, m), 2.78 (3H, d, J=4.5 Hz), 3.15-3.20 (1H, m), 3.75-3.82 (1H, m), 6.55-6.60 (1H, m), 6.71 (1H, d, J=7.9 Hz), 7.11 (1H, d, J=7.4 Hz), 7.39 (1H, d, J=11.2 Hz)

MS (ESI, m/z): 386 (M+Na)

### Preparation 63

The following compound was obtained in a similar manner to that of Preparation 7:

### tert-butyl {trans-4-[(3-fluoro-5-carbamoyl-6-methylaminopyridin-2-yl)amino]cyclohexyl}carbamate

1H-NMR (DMSO-d6): δ 1.19-1.40 (4H, m), 1.40 (9H, s), 1.75-1.85 (2H, m), 1.90-1.95 (2H, m), 2.83 (3H, d, J=4.8 Hz), 3.15-3.22 (1H, m), 3.75-3.83 (1H, m), 6.66-7.00 (4H, m), 7.64 (1H, d, J=12.8 Hz), 8.64 (1H, dd, J=4.7, 9.5 Hz)

MS (ESI, m/z): 404 (M+Na)

### Example 25

The following compound was obtained in a similar manner to that of Example 1:

### 6-[(trans-4-aminocyclohexyl)amino]-2-methylamino-5-fluoronicotinamide

1H-NMR (DMSO-d6): δ1.33-1.48 (4H, m), 1.95-2.15 (4H, m), 2.85 (3H, s), 2.95-3.05 (1H, m), 3.80-3.90 (1H, m), 6.00-7.00 (3H, m), 7.67 (1H, d, J=12.8 Hz), 8.02 (3H, br m)

MS (ESI, m/z): 282 (M+H)

### Preparation 64

The following compound was obtained in a similar manner to that of Preparation 1:

### tert-butyl {trans-4-[(3-fluoro-5-carbamoyl-6-chloropyridin-2-yl)amino]cyclohexyl}carbamate

1H-NMR (DMSO-d6): δ 1.20-1.35 (4H, m), 1.38 (9H, s), 1.7-1.9 (4H, m), 3.1-3.25 (1H, m), 3.65-3.8 (1H, m), 6.75 (1H, d, J=8.1 Hz), 7.16 (1H, d, J=7.7 Hz), 7.46 and 7.63 (2H, brs), 7.52 (1H, d, J=10.9 Hz).

MS (ESI, m/z): 409 (M+Na)

### Preparation 65

The following compound was obtained in a similar manner to that of Preparation 7:

### tert-butyl {trans-4-[(3-fluoro-5-carbamoyl-6-dimethylaminopyridin-2-yl)amino]cyclohexyl}carbamate

1H-NMR (DMSO-d6): δ 1.20-1.40 (4H, m), 1.40 (9H, s), 1.79-1.82 (2H, m), 1.92-1.95 (2H, m), 2.73 (6H, s), 3.20 (1H, m), 3.76 (1H, m), 6.71 (2H, m), 7.20 (1H, m), 7.45 (1H, d, J=11.7 Hz), 7.72 (1H, m)

MS (ESI, m/z): 418 (M+Na)

### Example 26

The following compound was obtained in a similar manner to that of Preparation 1:

### 6-[(trans-4-aminocyclohexyl)amino]-2-dimethylamino-5-fluoronicotinamide

1H-NMR (DMSO-d6): δ1.38-1.53 (4H, m), 1.98-2.04 (4H, m), 2.95-3.05 (1H, m), 3.02 (6H, s), 3.66 (1H, br s), 7.72 (1H, br s), 8.08 (1H, d, J=11.6 Hz), 8.33 (3H, m), 8.51 (1H, m) MS (ESI, m/z): 296 (M+H)

### Example 27

To a solution of 6-[(trans-4-aminocyclohexyl)amino]-2-methoxynicotinamide (100 mg) and triethylamine (46 mg) in dichloromethane (3 mL) was added dropwise methanesulfonyl chloride (52 mg) at ambient temperature and the mixture was stirred at the same temperature for 30 minutes. To the mixture was added methanol (10 mL) and evaporated in vacuo. The residue was purified by column chromatography on silica gel eluting with chloroform:methanol (5:1) to afford 6-[(trans-4-methanesulfonylaminocyclohexyl)amino]-2-methoxynicotinamide (128 mg) as white crystals.

1H-NMR (DMSO-d6): δ1.25-1.45 (4H, m), 1.9-2.1 (4H, m), 2.92 (3H, s), 3.1-3.2 (1H, m), 3.6-3.7 (1H, m), 3.91 (3H, s), 6.09 (1H, d, J=8.5 Hz), 6.97 (1H, d, J=7.3 Hz), 7.02 (1H, brs), 7.07 (1H, brs), 7.25 (1H, brs), 7.87 (1H, d, J=8.5 Hz)

MS (ESI, m/z): 365 (M+Na), 343 (M+H)

### Example 28

The following compound was obtained in a similar manner to that of Example 27:

### 6-[(trans-4-methanesulfonylaminocyclohexyl) amino]-5-fluoro-2-methoxynicotinamide

1H-NMR (DMSO-d6): δ1.27-1.50 (4H, m), 1.92-2.00 (4H, m), 2.92 (3H, s), 3.06-3.15 (1H, m), 3.72-3.84 (1H, m), 3.92 (3H, s), 7.01 (1H, bs), 7.14 (1H, d, J=6.8 Hz), 7.31 (1H, bs), 7.36 (1H, bs), 7.69 (1H, d, J=11.6 Hz)

MS (ESI, m/z): 383 (M+Na)

### Example 29

To a solution of 6-[(trans-4-aminocyclohexyl)amino]-5-fluoro-2-methoxy nicotinamide (200 mg) in methanol (30 mL) was added methyl vinyl sulfone (68 mg) and the mixture was stirred at ambient temperature for 3 hours. The mixture was evaporated in vacuo and the residue was purified by column chromatography on silica gel eluting with chloroform:methanol (5:1) to afford 6-[(trans-4-(2-methanesulfonyl)ethylaminocyclohexyl)amino]-5-fluoro-2-methoxynicotinamide (172 mg) as a powder.

1H-NMR (DMSO-d6): δ1.04-1.16 (2H, m), 1.32-1.44 (2H, m), 1.79 (1H, bs), 1.88-1.98 (4H, m), 2.32-2.43 (1H, m), 2.95 (2H, t, J=6.6 Hz), 3.01 (3H, s), 3.18 (2H, t, J=6.6 Hz), 3.77-3.88 (1H, m), 3.91 (3H, s), 7.12 (1H, d, J=6.7 Hz), 7.30 (1H, bs), 7.35 (1H, bs), 7.68 (1H, d, J=11.7 Hz)

MS (ESI, m/z): 389 (M+H)

### Example 30

To a solution of 6-[(trans-4-aminocyclohexyl)amino]-5-fluoro-2-methoxynicotinamide (200 mg) and tetrahydro-4H-thiopyran-4-one 1,1-dioxide (210 mg) in methanol (4 mL) was added sodium cyanoborohydride (89 mg) and the mixture was stirred at ambient temperature for 3 days. The mixture was concentrated in vacuo and poured into ethyl acetate and saturated sodium bicarbonate aqueous solution. The organic layer was washed with brine, dried over sodium sulfate and evaporated in vacuo. The residue was purified by column chromatography on silica gel eluting with chloroform:methanol (5:1) to afford 6-({trans-4-[(1,1-dioxidotetrahydro-2H-thiopyran-4-yl)amino]cyclohexyl}amino)-5-fluoro-2-methoxynicotinamide (108 mg) as a powder.

1H-NMR (DMSO-d6): δ 1.02-1.14 (2H, m), 1.30-1.43 (2H, m), 1.59 (1H, bs), 1.73-1.84 (2H, m), 1.86-2.07 (7H, m), 2.38-2.48 (1H, m), 2.87-2.96 (1H, m), 2.97-3.14 (4H, m), 3.76-3.88 (1H, m), 3.91 (3H, s), 7.13 (1H, d, J=7.4 Hz), 7.31 (bsH, 1), 7.35 (1H, bs), 7.68 (1H, d, J=11.7 Hz).

MS (ESI, m/z): 437 (M+Na)

### Example 31

The following compound was obtained in a similar manner to that of Example 30:

### 6-({trans-4-[(1,1-dioxidotetrahydro-2H-thiopyran-4-yl)amino]cyclohexyl}amino)-2,5-difluoronicotinamide

1H-NMR (DMSO-d6): δ1.00-1.12 (2H, m), 1.31-1.44 (2H, m), 1.73-1.94 (6H, m), 1.98-2.07 (2H, m), 2.34-2.46 (1H, m), 2.86-2.96 (1H, m), 2.97-3.14 (4H, m), 3.66-3.78 (1H, m), 7.23 (1H, bs), 7.38-7.48 (2H, m), 7.69 (1H, dd, J=7.4, 10.8 Hz)

MS (ESI, m/z): 425 (M+Na)

### Example 32

The following compound was obtained in a similar manner to that of Example 30:

### 6-({trans-4-[(1,1-dioxidotetrahydro-2H-thiopyran-4-yl)amino]cyclohexyl}amino)-2-chloro-5-fluoronicotinamide

1H-NMR (DMSO-d6): δ 1.00-1.13 (2H, m), 1.29-1.42 (2H, m), 1.59 (1H, bs), 1.73-1.96 (6H, m), 1.97-2.07 (2H, m), 2.35-2.45 (1H, m), 2.87-2.96 (1H, m), 2.97-3.14 (4H, m), 3.70-3.86 (1H, m), 7.14 (1H, d, J=8.0 Hz), 7.45 (1H, bs), 7.52 (1H, d, J=10.9 Hz), 7.62 (1H, bs)

MS (ESI, m/z): 419 (M+H)

### Preparation 66

A solution of 6-[(trans-4-aminocyclohexyl)amino]-5-fluoro-2-methoxy nicotinamide (700 mg) and 1H-benzotriazol-1-methanol (370 mg) in methanol (10 mL) was stirred at ambient temperature overnight. After removal of the solvent by evaporation, the residue was triturated with diisopropylether and collected by filtration. The powder was dissolved into ethanol (15 ml) and was added sodium borohydride (206 mg) in small portions over 1 hour at 5° C. After stirred for 3 hours, the mixture was evaporated and thereto were added dichloromethane (30 ml) and saturated sodium bicarbonate aqueous solution (30 mL). The whole was stirred vigorously and thereto was added di-tert-butyl carbonate (1.0 g) and the whole was stirred overnight. The resulting insoluble material was filtered off and the organic layer of the filtrate was separated and dried over magnesium sulfate, and evaporated. The residue was purified by column chromatography on silica gel eluting with chloroform:methanol (40:1). The fractions containing the objective compound were collected and evaporated. (The NMR data suggested the fraction is 3:2 mixture of each boc analog of starting material and the objective material.) The mixture (300 mg) was purified by recycling preparative HPLC equipped with gel-permeation chromatography column (chloroform as eluent) over 2 days, the separated fractions containing the objective material were collected to afford tert-butyl {trans-4-[(5-carbamoyl-3-fluoro-6-methoxypyridin-2-yl)amino]cyclohexyl}methylcarbamate (100 mg).

1H-NMR (DMSO-d6): δ 1.38-1.45 (2H, m), 1.40 (9H, s), 1.55-1.65 (4H, m), 1.99-2.05 (2H, m), 2.69 (3H, s), 3.70-3.86 (2H, m), 3.92 (3H, s), 7.13 (1H, d, J=7.3 Hz), 7.32 (1H, s), 7.36 (1H, s), 7.69 (1H, d, J=11.6 Hz).

MS (ESI, m/z): 419 (M+Na)

### Example 33

The following compound was obtained in a similar manner to that, of Example 13:

### 5-fluoro-2-methoxy-6-{[trans-4-(methylamino)cyclohexyl]amino}nicotinamide trifluoroacetate

1H-NMR (DMSO-d6): δ 1.35-1.45 (4H, m), 2.00-2.15 (4H, m), 2.57 (3H, s), 2.90-2.95 (1H, m), 3.80-2.90 (1H, m), 3.92 (3H, s), 7.21 (1H, d, J=8.5 Hz), 7.35 (1H, s), 7.37 (1H, s), 7.71 (1H, d, J=11.6 Hz), 8.15-8.35 (2H, m)

MS (ESI, m/z): 297 (M+H)

### Example 34

To a suspension of 6-[(trans-4-aminocyclohexyl)amino]-2-methoxynicotinamide (85 mg) and formalin (152 mg) was added sodium triacetoxyborohydride (409 mg) and the mixture was stirred at ambient temperature overnight. The mixture was evaporated in vacuo and the residue was purified by column chromatography on silica gel eluting with chloroform:methanol (4:1) to afford 6-{[trans-4-(dimethylamino)cyclohexyl]amino}-2-methoxynicotinamide (92 mg) as a white powder.

1H-NMR (DMSO-d6): δ1.14-1.34 (4H, m), 1.79-1.86 (2H, m), 2.0-2.06 (2H, m), 2.18 (6H, s), 3.91 (3H, s), 6.08 (1H, d, J=8.5 Hz), 7.03-7.09 (1H, m), 7.12 and 7.27 (total 2H, brs), 7.86 (1H, d J=8.5 Hz)

MS (ESI, m/z): 293 (M+H)

### Example 35

The following compound was obtained in a similar manner to that of Example 34:

### 6-{[trans-4-(dimethylamino)cyclohexyl]amino}-5-fluoro-2-methoxynicotinamide

1H-NMR (DMSO-d6): δ 1.21-1.41 (4H, m), 1.82-1.85 (2H, m), 1.97-2.00 (2H, m), 2.10 (1H, m), 2.17 (6H, s), 3.78 (1H, m), 3.92 (3H, s), 7.11 (1H, d, J=7.12 Hz), 7.31 (1H, s), 7.36 (1H, s), 7.68 (1H, d, J=11.7 Hz)

MS (ESI, m/z): 311 (M+H)

### Preparation 67

To a solution of 6-hydroxy-2-methoxynicotinonitrile (300 mg), tert-butyl (cis-4-hydroxycyclohexyl)carbamate (473 mg) and triphenylphosphine (786 mg) in tetrahydrofuran (9 mL) was diethyl azodicarboxylate (1.36 g) and the mixture was stirred at ambient temperature for 4 days. After removal of the solvent by evaporation, the residue was purified by column chromatography on silica gel eluting with hexane:ethyl acetate (1:1) to afford tert-butyl {trans-4-[(5-cyano-6-methoxypyridin-2-yl)oxy]cyclohexyl}carbamate (132 mg).

1H-NMR (DMSO-d6): δ 1.27-1.55 (4H, m), 1.38 (9H, s), 1.79-1.88 (2H, m), 2.06-2.15 (2H, m), 3.22-3.36 (1H, m), 3.96 (3H, s), 4.86-4.95 (1H, m), 6.49 (1H, d, J=8.4 Hz), 6.79 (1H, d, J=7.5 Hz), 8.04 (1H, d, J=8.4 Hz)

MS (ESI, m/z): 370 (M+Na)

### Preparation 68

The following compound was obtained in a similar manner to that of Preparation 7:

### tert-butyl {trans-4-[(5-carbamoyl-6-methoxypyridin-2-yl)oxy]cyclohexyl}carbamate

1H-NMR (DMSO-d6): δ 1.28-1.52 (4H, m), 1.38 (9H, s), 1.80-1.89 (2H, m), 2.07-2.16 (2H, m), 3.23-3.36 (1H, m), 3.97 (3H, s), 4.83-4.91 (1H, m), 6.42 (1H, d, J=8.2 Hz), 6.78 (1H, d, J=7.4 Hz), 7.47 (2H, bs), 8.14 (1H, d, J=8.2 Hz)

MS (ESI, m/z): 388 (M+Na)

### Example 36

The following compound was obtained in a similar manner to that of Example 13:

### 6-[(trans-4-aminocyclohexyl)oxy]-2-methoxynicotinamide trifluoroacetate

1H-NMR (DMSO-d6): δ 1.35-1.45 (4H, m), 2.00-2.15 (4H, m), 2.57 (3H, s), 2.90-2.95 (1H, m), 3.80-2.90 (1H, m), 3.92 (3H, s), 7.21 (1H, d, J=8.5 Hz), 7.35 (1H, s), 7.37 (1H, s), 7.71 (1H, d, J=11.6 Hz), 8.15-8.35 (2H, m)

MS (ESI, m/z): 297 (M+H)

### Preparation 69

The following compound was obtained in a similar manner to that of Preparation 1:

### tert-butyl 4-{[(5-cyanopyridin-2-yl)amino]methyl}piperidine-1-carboxylate

1H-NMR (DMSO-d6): δ 0.96-1.08 (2H, m), 1.38 (9H, s), 1.49-1.72 (4H, m), 2.66 (1H, brs), 3.20 (2H, brs), 3.91 (2H, brs), 6.55 (1H, d, J=9.42 Hz), 7.63-7.70 (2H, m), 8.37 (1H, d, J=2.07 Hz)

### Preparation 70

The following compound was obtained in a similar manner to that of Preparation 7:

### tert-butyl 4-{[(5-carbamoylpyridin-2-yl)amino]methyl}piperidine-1-carboxylate

1H-NMR (DMSO-d6): δ 0.94-1.08 (2H, m), 1.39 (9H, s), 1.67 (4H, d, J=10.9 Hz), 2.67 (1H, brs), 3.18 (2H, t, J=5.8 Hz), 3.93 (2H, d, J=12.3 Hz), 6.45 (1H, d, J=8.8 Hz), 7.01 (1H, brs), 7.13 (1H, t, J=5.1 Hz), 6.59 (1H, brs), 7.76-7.80 (1H, m), 8.49 (1H, d, J=2.2 Hz)

### Example 37

The following compound was obtained in a similar manner to that of Example 4:

### 6-[(piperidin-4-ylmethyl)amino]nicotinamide

1H-NMR (DMSO-d6): δ 0.95-1.08 (2H, m), 1.61 (3H, d, J=10.4 Hz), 2.38 (2H, t, J=12.3 Hz), 2.90 (2H, d, J=12.3 Hz), 3.14 (2H, t, J=5.6 Hz), 6.44 (1H, d, J=8.8 Hz), 7.01 (1H, brs), 7.08 (1H, t, J=5.6 Hz), 7.63 (1H, brs), 7.77 (1H, dd, J=8.8, 2.4 Hz), 8.49 (1H, d, J=2.4 Hz)

### Preparation 71

The following compound was obtained in a similar manner to that of Preparation 1:

### tert-butyl 4-[(5-cyanopyridin-2-yl)amino]piperidine-1-carboxylate

1H-NMR (DMSO-d6): δ 1.22-1.37 (2H, m), 1.39 (9H, s), 1.78 (2H, d, J=12.9 Hz), 3.05 (2H, t, J=12.9 Hz), 3.55 (1H, brs), 4.32 (2H, d, J=12.9 Hz), 6.88 (1H, d, J=7.9 Hz), 6.93 (1H, d, J=10.7 Hz), 7.81 (1H; dd, J=8.9, 2.3 Hz), 8.46 (1H, d, J=1.7 Hz)

### Preparation 72

The following compound was obtained in a similar manner to that of Preparation 7:

### tert-butyl 4-[(5-carbamoylpyridin-2-yl)amino]piperidine-1-carboxylate

1H-NMR (DMSO-d6): δ 1.23-1.38 (2H, m), 1.38 (9H, s), 1.77 (2H, d, J=10.2 Hz), 2.97 (2H, t, J=12.5 Hz), 3.51 (1H, brs), 4.31 (2H, d, J=13.6 Hz), 6.82-6.87 (2H, m), 7.11 (1H, brs), 7.74 (1H, brs), 7.92 (1H, dd, J=8.8, 2.8 Hz), 8.59 (1H, d, J=2.0 Hz)

### Example 38

The following compound was obtained in a similar manner to that of Example 4:

### 6-(piperidin-4-ylamino)nicotinamide

1H-NMR (DMSO-d6): δ 1.08-1.21 (2H, m), 1.74 (2H, d, J=12.9 Hz), 2.76-2.85 (1H, m), 2.96 (2H, t, J=10.7 Hz), 4.26 (2H, d, J=12.9 Hz), 6.82 (1H, d, J=8.8 Hz), 7.10 (1H, brs), 7.73 (1H, brs), 7.91 (1H, dd, J=9.5, 2.7 Hz), 8.58 (1H, d, J=3.4 Hz)

### Preparation 73

The following compound was obtained in a similar manner to that of Preparation 1:

### tert-butyl {4-[(5-cyanopyridin-2-yl)amino]butyl}carbamate

1H-NMR (DMSO-d6): δ 1.39 (9H, s), 1.39-1.53 (4H, m), 2.89-2.95 (2H, m), 3.24-3.30 (2H, m), 6.53 (1H, d, J=8.8 Hz), 6.79-6.84 (1H, m), 7.58-7.66 (2H, m), 8.37 (1H, d, J=2.0 Hz)

### Preparation 74

The following compound was obtained in a similar manner to that of Preparation 7:

### tert-butyl {4-[(5-carbamoylpyridin-2-yl)amino]butyl}carbamate

1H-NMR (DMSO-d6): δ 1.37 (9H, s), 1.38-1.52 (4H, m), 2.90-2.95 (2H, m), 3.21-3.29 (2H, m), 6.41 (1H, d, J=9.5 Hz), 6.79-6.84 (1H, m), 6.99-7.06 (2H, m), 7.63 (1H, brs), 7.78 (1H, dd, J=9.2, 2.2 Hz), 8.50 (1H, d, J=2.2 Hz)

### Example 39

The following compound was obtained in a similar manner to that of Example 4:

### 6-[(4-aminobutyl)amino]nicotinamide

1H-NMR (DMSO-d6): δ 1.31-1.56 (4H, m), 2.52 (2H, t, J=6.9 Hz), 3.20-3.24 (2H, m), 6.40 (1H, d, J=8.7 Hz), 6.99 (1H, s), 7.04 (1H, t, J=5.3 Hz), 7.63 (1H, s), 7.76 (1H, d, J=9.3 Hz), 8.50 (1H, s)

### Preparation 75

The following compound was obtained in a similar manner to that of Preparation 1:

### methyl 5-({trans-4-[(tert-butoxycarbonyl)amino]cyclohexyl}amino)pyrazine-2-carboxylate

1H-NMR (DMSO-d6): δ 1.26 (4H, t, J=8.4 Hz), 1.38 (9H, s), 1.80 (2H, brs), 1.92 (2H, brs), 3.20-3.35 (1H, m), 3.65-3.78 (1H, m), 3.78 (3H, s), 6.77 (1H, d, J=8.4 Hz), 7.91 (2H, s), 8.54 (1H, s)

### Preparation 76

The following compound was obtained in a similar manner to that of Preparation 3:

### 5-({trans-4-[(tert-butoxycarbonyl)amino]cyclohexyl}amino)pyrazine-2-carboxylic acid

1H-NMR (DMSO-d6): δ 1.26 (4H, t, J=9.45 Hz), 1.38 (9H, s), 1.80 (2H, brs), 1.94 (2H, brs), 3.24 (1H, brs), 3.66 (1H, brs), 6.76 (1H, d, J=8.1 Hz), 7.58 (1H, d, J=7.5 Hz), 7.85 (1H, s), 8.52 (1H, s)

### Preparation 77

The following compound was obtained in a similar manner to that of Preparation 4:

### tert-butyl {trans-4-[(5-carbamoylpyrazin-2-yl)amino]cyclohexyl}carbamate

1H-NMR (DMSO-d6): δ 1.21-1.27 (4H, m), 1.38 (9H, s), 1.80 (2H, brs), 1.95 (2H, brs), 3.17-3.31 (1H, m), 3.60-3.74 (1H, m), 6.77 (1H, d, J=8.1 Hz), 7.27 (1H, s), 7.60 (1H, s), 7.63 (1H, s), 7.83 (1H, s), 8.50 (1H, s)

### Example 40

The following compound was obtained in a similar manner to that of Example 4:

### 5-[(trans-4-aminocyclohexyl) amino]pyrazine-2-carboxamide

1H-NMR (DMSO-d6): δ 1.09-1.29 (4H, m), 1.43 (1H, brs), 1.77 (2H, d, J=11.7 Hz), 1.92 (2H, d, J=12.3 Hz), 3.68 (1H, brs), 7.26 (1H, s), 7.56 (1H, d, J=7.8 Hz), 7.62 (1H, s), 7.82 (1H, s), 8.50 (1H, s)

### Preparation 78

The following compound was obtained in a similar manner to that of Preparation 1:

### methyl 3-amino-5-({trans-4-[(tert-butoxycarbonyl)amino]cyclohexyl}amino)-6-chloropyrazine-2-carboxylate

1H-NMR (DMSO-d6): δ 1.1-1.3 (2H, m), 1.38 (9H, s), 1.4-1.6 (2H, m), 1.75-1.9 (4H, m), 3.1-3.25 (1H, m), 3.8-3.95 (1H, m), 6.78 (1H, d, J=8.0 Hz), 7.15 (1H, d, J=8.0 Hz), 7.28 (2H, brs)

### Preparation 79

The following compound was obtained in a similar manner to that of Preparation 3:

### 3-amino-5-({trans-4-[(tert-butoxycarbonyl)amino]cyclohexyl}amino)-6-chloropyrazine-2-carboxylic acid

1H-NMR (DMSO-d6): δ 1.2-1.5 (4H, m), 1.38 (9H, s), 1.7-1.9 (4H, m), 3.15-3.3 (1H, m), 3.7-3.85 (1H, m), 6.00 (1H, d, J=6.2 Hz), 6.75 (1H, d, J=6.2 Hz)

MS (ESI, m/z): 408 (M+Na)

### Preparation 80

The following compound was obtained in a similar manner to that of Preparation 4:

### tert-butyl {trans-4-[(6-amino-5-carbamoyl-3-chloropyrazin-2-yl)amino]cyclohexyl}carbamate

1H-NMR (DMSO-d6): δ 1.15-1.34 (2H, m), 1.38 (9H, s), 1.42-1.55 (2H, m), 1.78 (2H, brs), 1.81 (2H, brs), 3.16-3.24 (1H, m), 3.81-3.87 (1H, m), 6.80 (2H, q, J=7.7 Hz), 7.01 (1H, 7.27 (1H, s)

### Example 41

The following compound was obtained in a similar manner to that of Example 4:

### 3-amino-5-[(trans-4-aminocyclohexyl)amino]-6-chloropyrazine-2-carboxamide

1H-NMR (DMSO-d6): δ 1.21-1.33 (2H, m), 1.43-1.51 (2H, m), 1.87 (4H, t, J=12.2 Hz), 2.80-2.86 (1H, m), 3.74-3.90 (1H, m), 6.82 (1H, d, J=7.8 Hz), 7.04 (1H, s), 7.28 (1H, s)

### Preparation 81

The following compound was obtained in a similar manner to that of Preparation 4:

### tert-butyl {trans-4-[(4-amino-5-cyanopyrimidin-2-yl)amino]cyclohexyl}carbamate

1H-NMR (DMSO-d6): δ 1.15-1.3 (4H, m), 1.38 (9H, s), 1.75-1.85 (4H, m), 3.1-3.2 (1H, m), 3.6-3.7 (1H, m), 6.7-6.8 (1H, m), 7.15-7.25 (1H, m), 8.12 (1H, s)

### Example 42

tert-butyl {trans-4-[(4-amino-5-cyanopyrimidin-2-yl)amino]cyclohexyl}carbamate (1.03 g) was suspended in concentrated sulfuric acid (14.1 g) and the mixture was stirred at 80° C. for 3 hours. The resultant solution was cooled to 5° C. and poured into ice-water (50 mL). The mixture was adjusted to pH 10 by addition of 5N sodium hydroxide aqueous solution under cooling and extracted with ethyl acetate. The organic layer was washed with brine, dried over magnesium sulfate, and evaporated in vacuo. The residue was purified by column chromatography on NH silica gel (Fuji Silysia chemical Ltd.) eluting with chloroform:methanol (10:1) to afford 4-amino-2-[(trans-4-aminocyclohexyl)amino]pyrimidine-5-carboxamide (280 mg) as a pale-yellow powder.

1H-NMR (DMSO-d6): δ 1.0-1.3 (4H, m), 1.65-1.85 (4H, m), 3.4-3.6 (2H, m), 6.6-6.9 (2H, m), 8.36 (1H, s)

TABLE 1
example number and its chemical compound

| Ex | Str. |
|---|---|
| 1 | 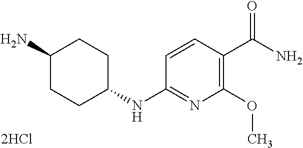 |
| 2 | 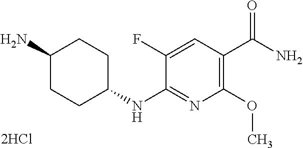 |
| 3 | 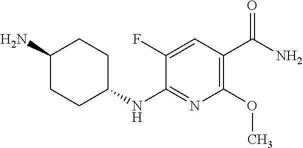 |
| 4 | 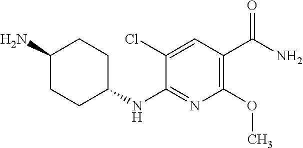 |
| 5 | 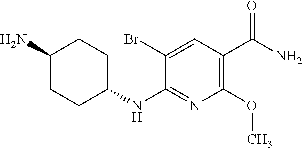 |
| 6 | 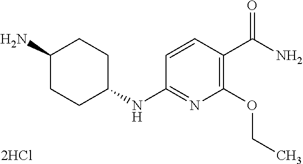 |
| 7 | 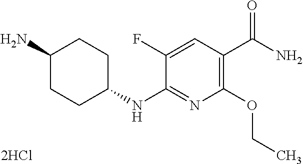 |
| 8 | 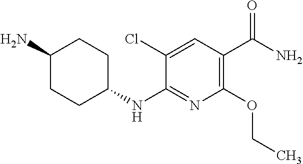 |
| 9 | 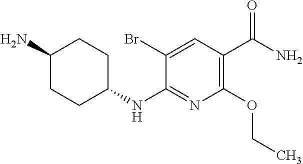 |
| 10 | 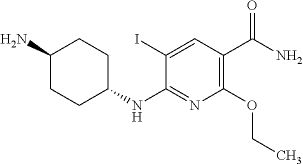 |
| 11 | 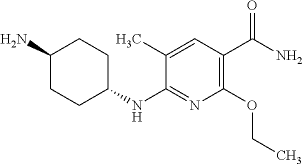 |
| 12 | 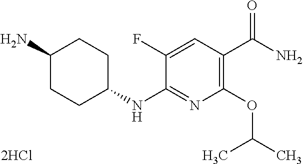 |
| 13 | 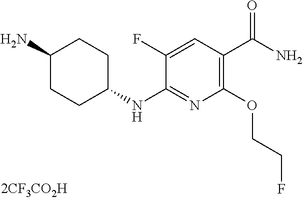 |
| 14 | 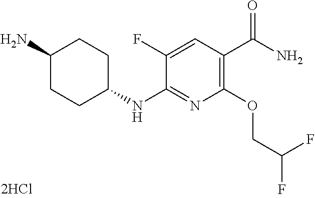 |
| 15 | 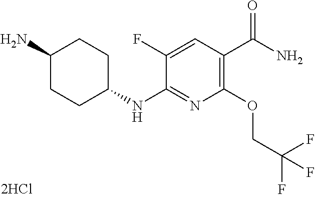 |
| 16 | 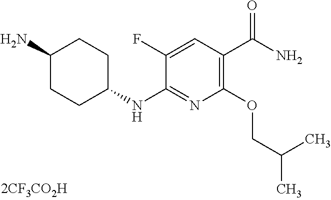 |
| 17 | 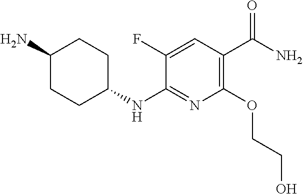 |
| 18 | 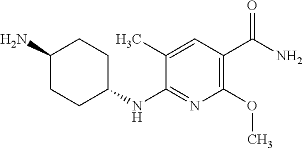 |
| 19 | 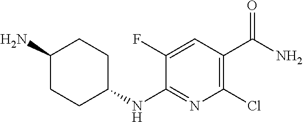 |
| 20 | 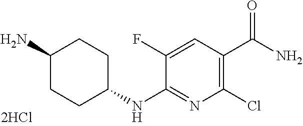 |
| 21 | 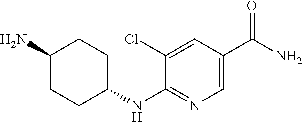 |
| 22 | 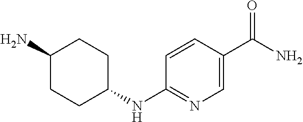 |
| 23 | 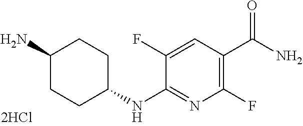 |
| 24 | 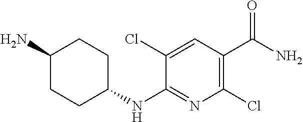 |
| 25 | 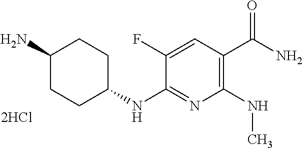 |
| 26 | 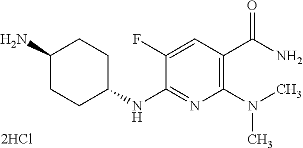 |
| 27 | 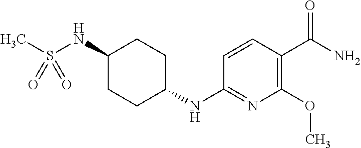 |
| 28 | 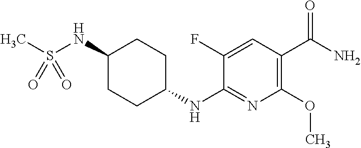 |
| 29 | 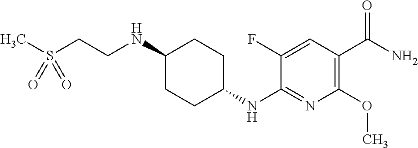 |
| 30 | 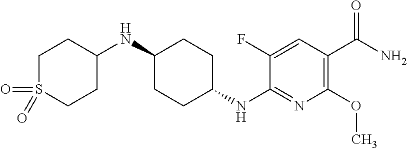 |
| 31 | 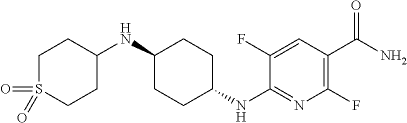 |
| 32 | 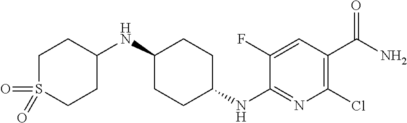 |
| 33 | 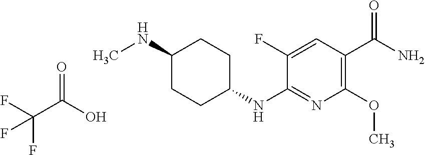 |
| 34 | 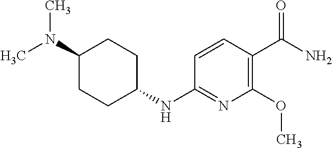 |
| 35 | 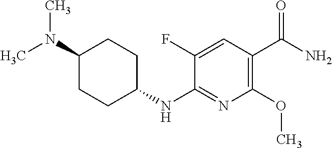 |
| 36 | 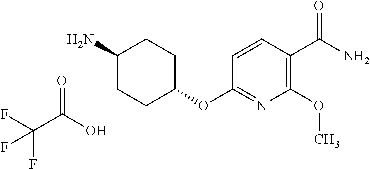 |
| 37 | 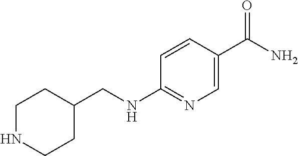 |
| 38 | 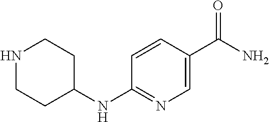 |
| 39 | 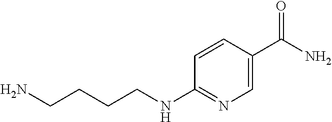 |
| 40 | 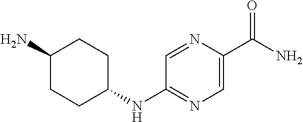 |
| 41 | 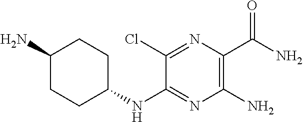 |
| 42 | 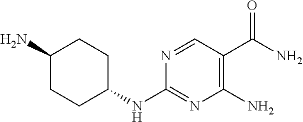 |

Ex: example number;
Str.: chemical structure;

### Pharmacological Assay

In order to show the usefulness of the compound [I] for the prophylactic and therapeutic treatment of above-mentioned disease in human being or animals, some representative compounds in the compounds [I] of the present invention as shown in the above examples were tested on the ROCK-inhibiting activity as follows.

### 1. ROCK Enzyme Inhibition Assay

ROCK enzyme inhibitory activity of the compounds of the present invention has been assayed as follow. An aqueous solution of Rho kinase substrate MYTP was added to 96-well plate. Following incubation for overnight at 4° C., the plate was blocked by using blocking buffer containing BSA. To the plate was added reaction buffer containing each concentration of compound, suitable concentration of human ROCK I (Caruna Biosciences), ATP, β-glycerol phosphate, EGTA, sodium orthovanadate and DTT, and then the plate was incubated for 1 hour. After washing the plate by washing buffer, anti-phosphothreonin antibody was added to the plate, and then the plate was incubated for 1 hour. After washing the plate by washing buffer again, HRP-conjugated anti-phosphorylated protein antibody was added to the plate, and then the plate was incubated for 1 hour. After washing the plate by washing buffer again, colorimetric substrate TMB microwell peroxidase substrate was added to the plate, and then the plate was incubated for appropriate time. After incubation, sulfuric acid was added to the plate to stop the reaction, and then absorbance (450 nm) was measured by using spectromether. Based, on the absorbance for each compound, the data is fit using Prism software to obtain a IC₅₀ value.

TABLE 2
example number and its human ROCK I IC₅₀

| | human ROCK I | | human ROCK I |
|---|---|---|---|
| Compound | IC₅₀ (nM) | Compound | IC₅₀ (nM) |
| Example 1 | 2 | Example 6 | 10 |
| Example 2 | 2 | Example 7 | 9 |
| Example 4 | 5 | Example 28 | 58 |
| Example 5 | 39 | Example 30 | 7 |

### 2. Assessment of Changes in Hindlimb Weight Distribution by ROCK Inhibitor

As intra-articular injection of monosodium iodoacetate (MIA) induced human osteoarthritis-like histopathological changes in knee joint, this model would be very useful for the study of human osteoarthritis. Recently, it is reported the clinical scores of the joint pain were closely correlated to the grade of histological findings (J Vet Med Sci 65, 1195, 2003) and this model may be useful for estimation of therapeutic effects of pain with osteoarthritis.

Male SD rats were anesthetized with halothane (Takeda, Japan) and given single intra-articular injection of 1 mg of monosodium iodoacetate (MIA; Sigma, St. Louis, USA) through the infrapatellar ligament of the right knee. MIA was dissolved with physiologic saline and administered in a volume of 50 μl using a 27-gauge, 0.5-inch needle. Three weeks after the injection, hindlimb weight distribution was determined using an incapacitance tester (Linton Instrumentation, Norfork, UK). Rats were allowed to acclimate to the testing apparatus and when stationary, readings were taken over a 5 s period. Oral administration of chemical compounds restored the difference in the amount of weight between the left and right limbs. Each group in each experiment was performed in about 8 animals.

TABLE 3
example number and its weight distribution ED₅₀

| Compound | Weight distribution ED₅₀ (mg/kg) |
|---|---|
| Example 1 | <1 |
| Example 2 | <1 |
| Example 4 | <1 |
| Example 5 | <3 |

### 3. Assessment of Effects on Hindlimb Blood Flow by ROCK Inhibitor

As one of the study to clarify the usefulness of the compounds of the present invention for peripheral arterial disease, we conducted a study to evaluate the effects of the compounds on hindlimb blood flow in rats.

Male Wistar rats were anesthetized by intraperitoneal pentobarbital injection (Kanto chemical, Japan) after oral administration of compounds. Rats were placed on a heating plate for subsequent hindlimb blood flow analysis. We measured hindlimb blood flow using a laser Doppler blood flow meter (PeriScan System, Stockholm, Sweden). After the laser Doppler images were recorded, the average perfusion values of both limbs were calculated, and evaluated the effects of compounds on hindlimb blood flow. Each experiment was performed in about 4 animals.

TABLE 4
example number and its % of increase of blood flow

| Compound | % of increase of blood flow (10 mg/kg p.o.) |
|---|---|
| Example 1 | >130% |
| Example 2 | >130% |
| Example 4 | >130% |
| Example 5 | >130% |
| Example 6 | >130% |
| Example 7 | >130% |
| Example 28 | >130% |
| Example 30 | >130% |

### 4. Assessment of the Inhibitory Effects on Phenylephrine-Induced Elevation of Urethral Pressure by Rock Inhibitor

To evaluate the usefulness of the compounds of the present invention for urinary dysfunction associated with benign prostatic hyperplasia, we investigated the inhibitory effects of the intravenous administration of the compounds on phenylephrine-induced elevation of the urethral pressure in rats.

Male Wistar rats were anesthetized with urethane (SIGMA, USA). After midline abdominal incision, catheter to measure the urethral pressure (3.5 Fr, Millar, USA) was inserted into the urethra from top of the bladder. After confirming the Phenylephrine (30 μg/kg i.v.)-induced elevation of the urethral pressure, compounds were administered intravenously in increasing doses. Every 5 min after administration of compounds, Phenylephrine (30 μg/kg i.v.) was injected, and the inhibitory effects of compounds on phenylephrine-induced elevation of urethral pressure were evaluated. Each experiment was performed in about 4 animals.

TABLE 5
example number and its Inhibition of elevation in urethral
pressure ED₃₀

| | Inhibition of elevation in urethral |
|---|---|
| Compound | pressure ED₃₀ (mg/kg i.v.) |
| Example 1 | <0.5 |
| Example 2 | <0.5 |
| Example 4 | <0.5 |
| Example 5 | <0.5 |
| Example 6 | <0.5 |
| Example 7 | <0.5 |
| Example 28 | <0.5 |
| Example 30 | <0.5 |

### 5. Therapeutic Effect of the Rock Inhibitor in Bleomycin-Induced Lung Fibrosis Model

To induce pulmonary fibrosis, ten mg/kg bleomycin (Nipponkayaku Industries Ltd, Tokyo, Japan) was intraperitoneally injected into female C57Bl/6 mice (9 weeks old, Charles River Laboratories Japan, Inc.) once a day from day 1 to day 10. Bleomycin was dissolved in saline and control mice were intraperitoneally injected with saline. The compound or vehicle was orally administered to the mice once a day from day 1 to day 35. On day 36, mice were sacrificed under anesthesia of pentobarbital and the lung was surgically removed. Hydroxyproline content of the lung was assayed according to the method of Woessner JF (1). Amount of hydroxyproline was significantly increased by injections of bleomycin compared to those of saline. The hydroxyproline content significantly decreased by the treatment of the compound (Example 3) and 66% reduction was achieved at 1 mg/kg.

### 6. In Vivo Studies of Eye Pressure Lowering Effect

The intraocular pressure (IOP) was determined in beagle dogs and rats with tonometer (TONO-PEN™, MENTOR and TonoLab™, M. E. Technica), specially calibrated for the eye of the particular species. In beagle dogs, the cornea was anesthetized with oxibuprocain before each IOP measurement.

TABLE 6
Intraocular pressure lowering effects in animals. The
compounds were applied topically.

| Compound | Dose | IOP change in animals after 4 hours |
|---|---|---|
| Example 3 | 10 mM | −16% reduced in beagle dog |
| Example 9 | 10 mM | −30% reduced in rat |
| Example 10 | 10 mM | −26% reduced in rat |

### INDUSTRIAL APPLICABILITY

As mentioned above, the present invention can provide novel heterocyclic carboxamide derivatives and salts thereof, which act as a ROCK inhibitor, to a pharmaceutical composition comprising the same and to a method of using the same therapeutically in the treatment and/or prevention of ROCK-related disease.

1. A compound of the formula [I] or its pharmaceutically acceptable salt: 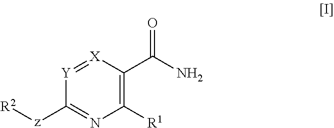
wherein
R¹ is hydrogen, halogen, optionally substituted lower alkyl, optionally substituted —O-lower alkyl, optionally substituted amino, or amino lower alkyl;
R² is cycloalkyl, heterocyclic group or lower alkyl, each of which may be optionally substituted;
X and Y are each N or CR³ in which R³ is hydrogen, halogen, lower alkyl, —O-lower alkyl, trifluoromethyl, or amino;
z is bond, —O—, or —NR⁴—, in which R⁴ is hydrogen, or optionally substituted lower alkyl.

2. The compound of claim 1, wherein
R¹ is hydrogen, halogen, —O-lower alkyl which may be substituted with halogen or —OH, or amino which may be substituted with lower alkyl;
R² is cycloalkyl which is substituted with optionally substituted amino, heterocyclic group, or amino lower alkyl;
X is CH or N;
Y is N or CR³ in which R³ is hydrogen, halogen or lower alkyl;
z is —O— or —NH—,
or its pharmaceutically acceptable salt.

3. The compound of claim 2, which is
R¹ is —O-lower alkyl which may, be substituted with halogen or —OH;
R² is cycloalkyl which is substituted with —NH₂, —NH-lower alkyl, —N(lower alkyl)₂, —NH—SO₂-lower alkyl or —NH-lower alkyl-SO₂-lower alkyl;
X is CH or N;
Y is N or CR³ in which R³ is hydrogen, halogen or lower alkyl;
z is —NH—,
or its pharmaceutically acceptable salt.

4. The compound of claim 3, which is
6-[(trans-4-aminocyclohexyl)amino]-5-fluoro-2-methoxynicotinamide,
or its pharmaceutically acceptable salt.

5. A pharmaceutical composition which comprises, as an active ingredient, a compound of claim 1, or a pharmaceutically acceptable salt thereof, in admixture with pharmaceutically, acceptable carriers or excipients.

6. Use of a compound of claim 1, or a pharmaceutically acceptable salt thereof, for the manufacture of a medicament.

7. Use of a compound of claim 1, or a pharmaceutically acceptable salt thereof, for the manufacture of a medicament for treating and/or preventing osteoarthritis, peripheral arterial disease, benign prostatic hypertrophy (BPH), idiopathic pulmonary fibrosis, glaucoma or ocular hypertension.

8. A ROCK inhibitor which comprises, as an active ingredient, a compound of claim 1, or a pharmaceutically acceptable salt thereof.

9. A method for treating and/or preventing ROCK-related disease, comprising administering a compound of claim 1, or a pharmaceutically acceptable salt thereof, to a patient in need of such treatment.

10. A method for treating and/or preventing ROCK-related disease selected from the group consisting of osteoarthritis, peripheral arterial disease, benign prostatic hypertrophy (BPH), idiopathic pulmonary fibrosis, glaucoma and ocular hypertension, comprising administering a compound of claim 1, or a pharmaceutically acceptable salt thereof, to a patient in need of such treatment.

11. A pharmaceutical composition for treating and/or preventing ROCK-related disease comprising a compound of claim 1, or a pharmaceutically acceptable salt thereof.

12. A pharmaceutical composition for treating and/or preventing ROCK-related disease selected from the group consisting of osteoarthritis, peripheral arterial disease, benign prostatic hypertrophy (BPH), idiopathic pulmonary fibrosis, glaucoma and ocular hypertension, comprising a compound of claim 1, or a pharmaceutically acceptable salt thereof.
